(19) United States
(12) Patent Application Publication
(10) Pub. No.: US 2019/0314421 A1
(43) Pub. Date: Oct. 17, 2019

(54) NUCLEIC ACID CONSTRUCTS INCLUDING A TXNIP PROMOTER FOR THE TREATMENT OF DISEASE

(71) Applicant: Wayne State University, Detroit, MI (US)

(72) Inventor: Lalit Singh Pukhrambam, West Bloomfield, MI (US)

(73) Assignee: Wayne State University, Detroit, MI (US)

(21) Appl. No.: 16/419,969

(22) Filed: May 22, 2019

Related U.S. Application Data
(63) Continuation of application No. 15/449,593, filed on Mar. 3, 2017.
(60) Provisional application No. 62/303,245, filed on Mar. 3, 2016.

(51) Int. Cl. A61K 35/35; C07K 14/65; C12N 15/11; C12N 15/113; C07K 14/62; A61K 9/00; C07K 14/47; C07K 14/475; C12N 9/02; C07K 14/705

(52) U.S. Cl. CPC A61K 35/35; A61K 38/00; C12N 15/111; C12Y 108/0401; C12N 15/1136; C07K 14/62; C12N 15/1135; A61K 9/0019; C07K 14/47; C07K 14/475; C12N 9/0075; C12N 9/0051; C07K 14/705; C12Y 114/13039; C12N 2330/51; C12N 2320/30; C12N 2310/14; C07K 14/65

(57) ABSTRACT

Nucleic acids for the treatment of diseases are described, as are cells including such nucleic acids and methods of using both nucleic acids and cells. The nucleic acids include a thioredoxin-interacting protein (TXNIP) promoter and a gene that encodes a therapeutic protein or an interfering nucleic acid sequence (e.g., interfering RNA (iRNA sequence)).

### CROSS-REFERENCE TO RELATED APPLICATION

This application is a continuation of co-pending U.S. patent application Ser. No. 15/449,593, filed Mar. 3, 2017, which claims priority to U.S. 62/303,245 filed on Mar. 3, 2016; both of which are incorporated herein by reference in their entirety as if fully set forth herein.

### STATEMENT OF GOVERNMENT SUPPORT

This invention was made with Government support under Grant No. RO1 EY023992 awarded by the National Institutes of Health National Eye Institute. The Government has certain rights in the invention.

### REFERENCE TO SEQUENCE LISTING

A computer readable text file, entitled “Sequence Listing.txt” created on or about May 22, 2019, with a file size of 96 KB, contains the sequence listing for this application and is hereby incorporated by reference in its entirety.

### FIELD OF THE DISCLOSURE

The current disclosure provides nucleic acids for the treatment of diseases. The nucleic acids include a thioredoxin-interacting protein (TXNIP) promoter and a gene that encodes a therapeutic protein or an interfering nucleic acid sequence.

### BACKGROUND OF THE DISCLOSURE

Diabetes mellitus (DM) is a group of metabolic diseases in which there is a high blood sugar level over a prolonged period. There are three main types of DM: Type 1 diabetes (insulin-dependent diabetes or childhood-onset diabetes), Type 2 diabetes (non-insulin-dependent diabetes or adult-onset diabetes), and gestational diabetes. Type 1 diabetes is caused by the autoimmune destruction of insulin producing beta-cells in the pancreas. Type 2 diabetes is caused by a combination of insulin resistance and inadequate insulin secretion. Gestational diabetes is a loss of blood sugar control that occurs during pregnancy and generally resolves after birth of the baby.

Current treatment of DM includes monitoring blood glucose levels and administering insulin when needed, administering oral hypoglycemic agents, and transplanting insulin-producing pancreatic beta-cells. Despite efforts to treat diabetes, it can nonetheless lead to many complications including diabetic ketoacidosis, nonketotic hyperosmolar coma, cardiovascular disease, stroke, chronic kidney failure, foot ulcers, and damage to the eyes.

Diabetic retinopathy (DR) is a severe complication of diabetes causing damage to the retina. It can eventually lead to blindness. DR affects up to 80 percent of all diabetic patients who have had diabetes for 20 years or more. DR accounts for 12% of all new cases of blindness each year in the United Stated and is the leading cause of blindness for people aged 20 to 64 years.

According to the International Diabetes Federation, the global population of individuals with diabetes was around 240 million is 2010, and is expected to rise to 300 million by 2025. The treatment of diabetes was estimated to cost 110 million dollars for 2011 and is expected to rise to almost 157 million dollars by 2017.

### SUMMARY OF THE DISCLOSURE

The current disclosure provides nucleic acids for the treatment of diseases. The nucleic acids include a thioredoxin-interacting protein (TXNIP) promoter and a gene that encodes a therapeutic protein or an interfering RNA (iRNA sequence).

In particular embodiments, the current disclosure provides nucleic acid constructs including a thioredoxin-interacting protein (TXNIP) promoter operably linked to a gene encoding (i) insulin or an insulin-like or insulin-promoting protein; (ii) a protein that reduces cellular oxidative stress, inflammation and/or apoptosis; and/or (iii) an interfering RNA sequence (iRNA) that reduces expression of a protein that promotes cellular oxidative stress, inflammation and/or apoptosis. The current disclosure also provides compositions including the nucleic acids for the treatment of diseases, and methods and kits utilizing the same.

A TXNIP promoter was chosen because TXNIP is a pro-apoptotic protein critically involved in the progression of diseases and their complications. For example, in relation to diabetes, the TXNIP promoter and TXNIP's associated expression is upregulated by high glucose within minutes. Thus, placing a TXNIP promoter in operable combination with a therapeutic gene of interest allows controlled administration of the therapeutic during times of hyperglycemia.

### BRIEF DESCRIPTION OF THE FIGURES

FIGS. 1A-1E provide exemplary sequences. FIG. 1A provides a thioredoxin interacting protein (TXNIP) promoter region including nucleotides −1 to −1526 of Gene ID: 117514 (SEQ ID NO: 1). FIG. 1B provides a human TXNIP promoter found at gene ID: 10628 (SEQ ID NO: 28);

FIG. 10 provides a mouse TXNIP promoter found at gene ID: 56338 (SEQ ID NO: 29). FIG. 1D provides a rheus monkey TXNIP promoter found at gene ID 698683 (SEQ ID NO: 30). FIG. 1E provides an exemplary thioredoxin 1 (Trx1) cDNA (FIG. 1B; GenBank: NM_053800.3; SEQ ID NO: 2).

FIGS. 2A-2D provide schematics of representative nucleic acid constructs disclosed herein (FIG. 2A); representative vector structures (FIG. 2B); and an exemplary nucleic acid construct (SEQ ID NO: 3, FIG. 2C) including a TXNIP promoter sequence (Gene ID: 117514) operably linked to an exemplary Trx1 cDNA sequence (GenBank: NM_053800.3; underlined in FIG. 2C; SEQ ID NO: 43). FIG. 2D provides SEQ ID NO: 31 including a TXNIP promoter+Thioredoxin 1 (Trx-1) cDNA with a length of 1939 bp and an additional 5′ sequence and 3′ sequence, both additional sequences underlined (Vector name: pUC57).

FIG. 3A provides an exemplary insulin sequence (GenBank: AAA59172.1; SEQ ID NO: 4). FIG. 3B provides an exemplary encoding cDNA sequence for human insulin mRNA (NM_000207.2; SEQ ID NO: 32).

FIG. 4A provides an exemplary insulin-like growth factor-1 (IGF-1) sequence (GenBank NP_001104753, SEQ ID NO: 5). FIG. 4B provides an exemplary encoding cDNA sequence for human IGF-1 (X00173.1; SEQ ID NO: 33).

FIG. 5A provides an exemplary pancreatic and duodenal homeobox 1 (PDX1) sequence (GenBank NP_000200, SEQ ID NO: 6). FIG. 5B provides an exemplary encoding cDNA sequence for human PDX1 (B0111592.2; SEQ ID NO: 34).

FIG. 6A provides an exemplary Trx1 sequence (GenBank AAF86466.1, SEQ ID NO: 7).

FIG. 6B provides an exemplary encoding cDNA sequence (AF276919.1; SEQ ID NO: 35).

FIG. 7A provides an exemplary thioredoxin 2 (Trx2) sequence (GenBank AAF86467.1, SEQ ID NO: 8). FIG. 7B provides an exemplary encoding cDNA sequence for Trx2 (AF276920.1; SEQ ID NO: 36).

FIG. 8A provides an exemplary TXNIP sequence (GenBank AAH93704.1, SEQ ID NO: 9). FIG. 8B provides an exemplary encoding cDNA sequence for TXNIP that can be targeted for down-regulation (B0093704.1; SEQ ID NO: 37).

FIG. 9A provides an exemplary vascular endothelial growth factor-A (VEGF-A) sequence (GenBank P15692.2, SEQ ID NO: 10). FIG. 9B provides an exemplary encoding cDNA sequence for VEGF-A that can be targeted for down-regulation (M32977.1; SEQ ID NO: 38).

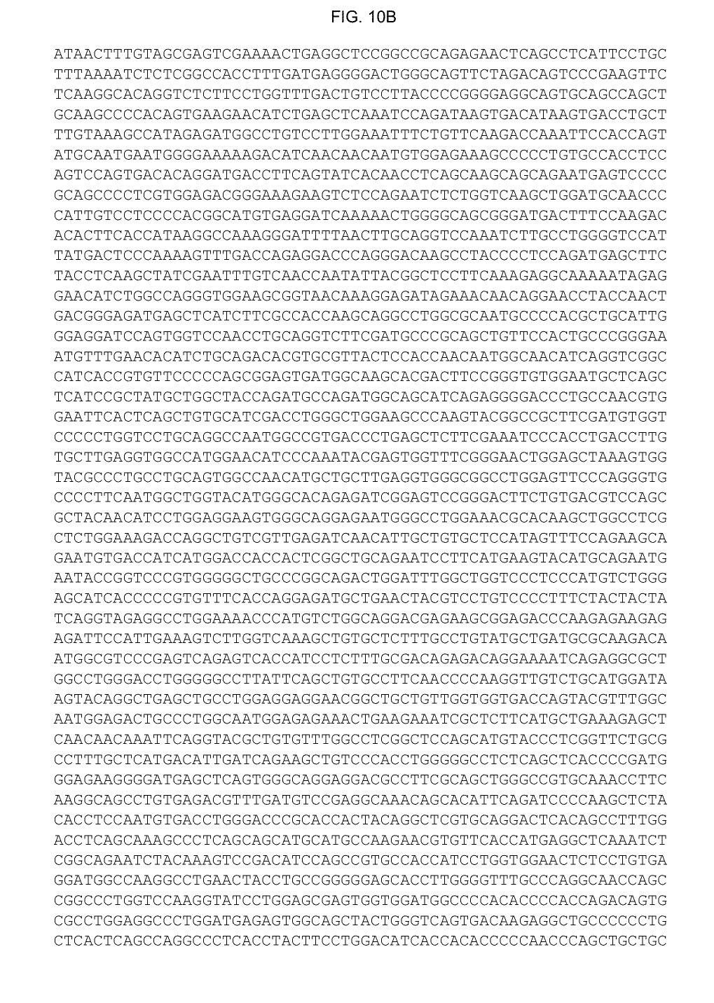

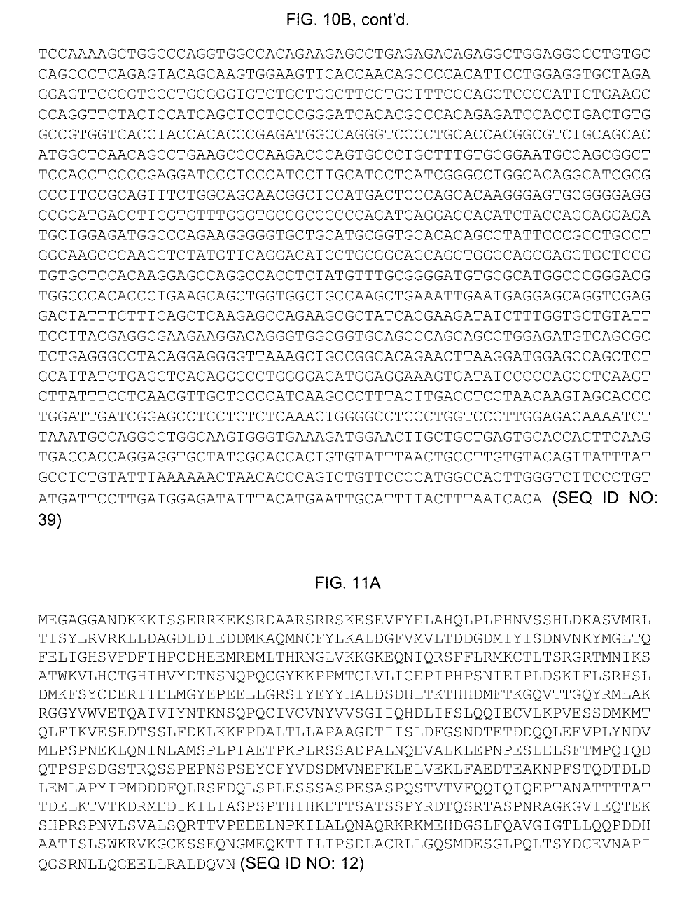

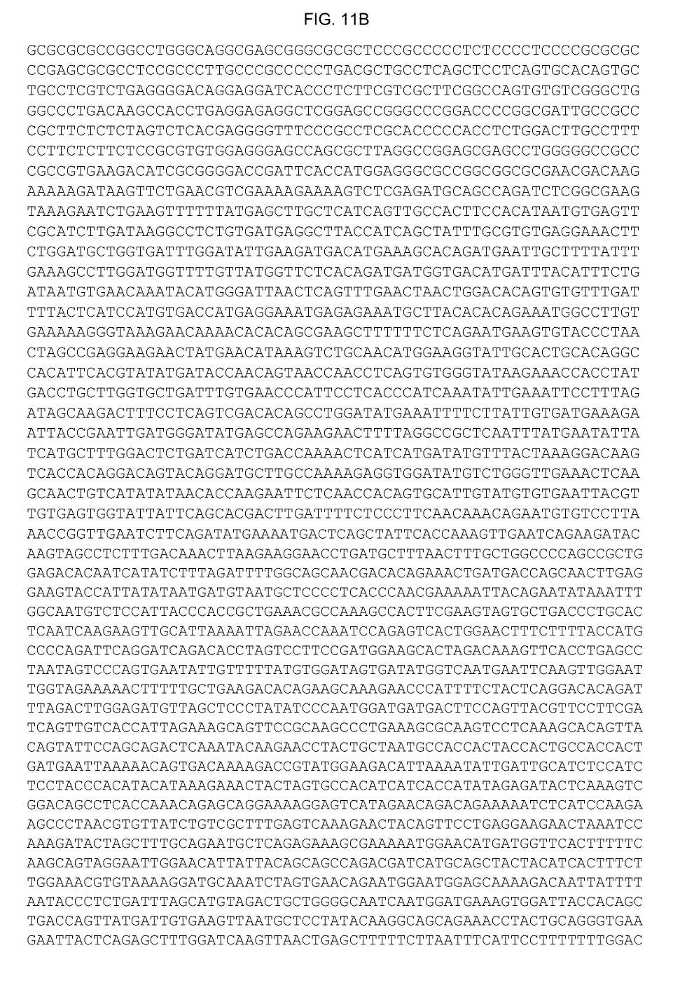

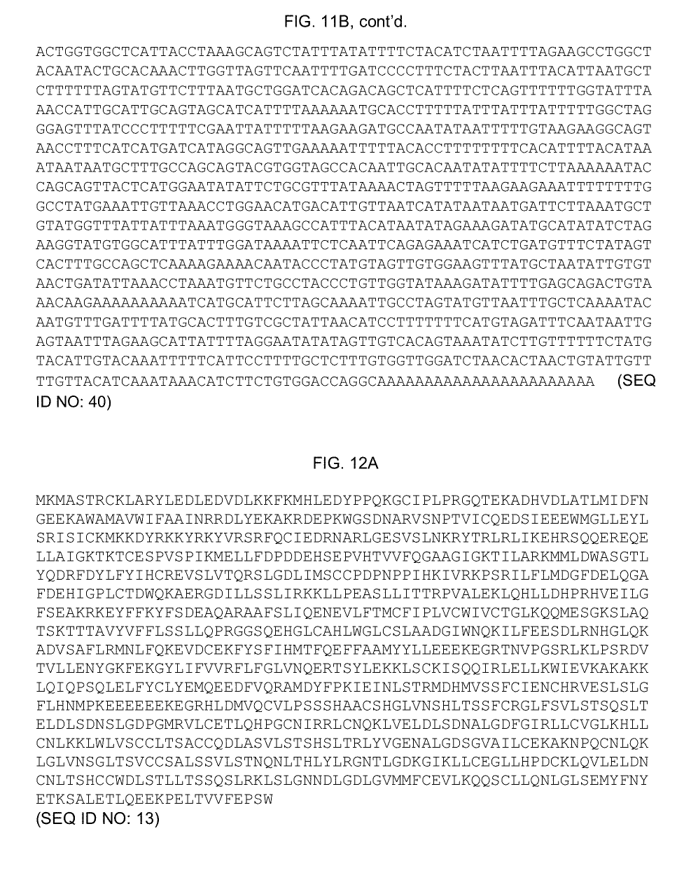

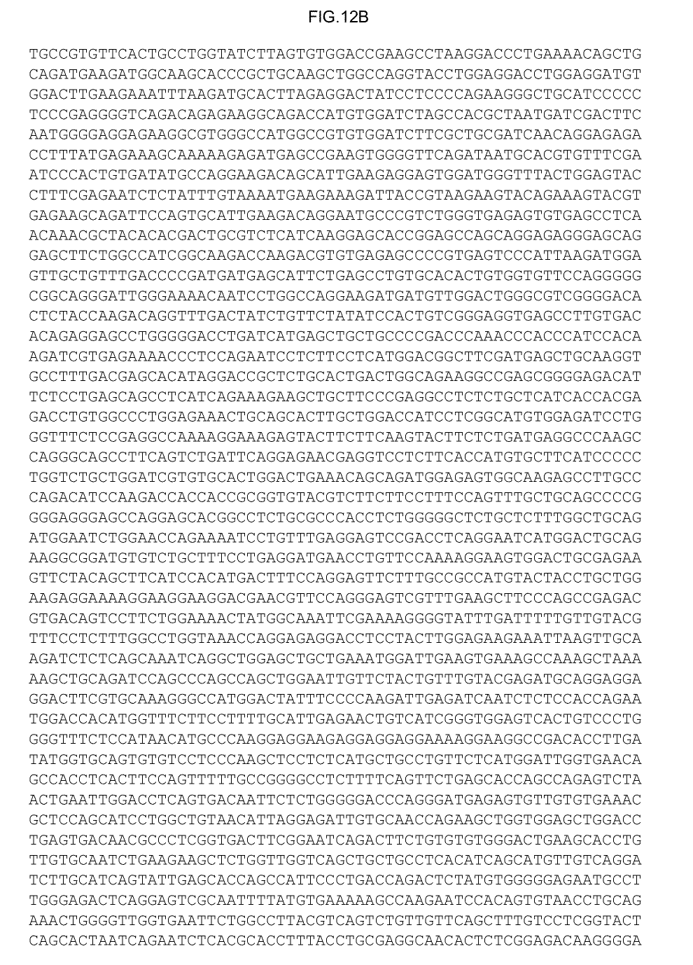

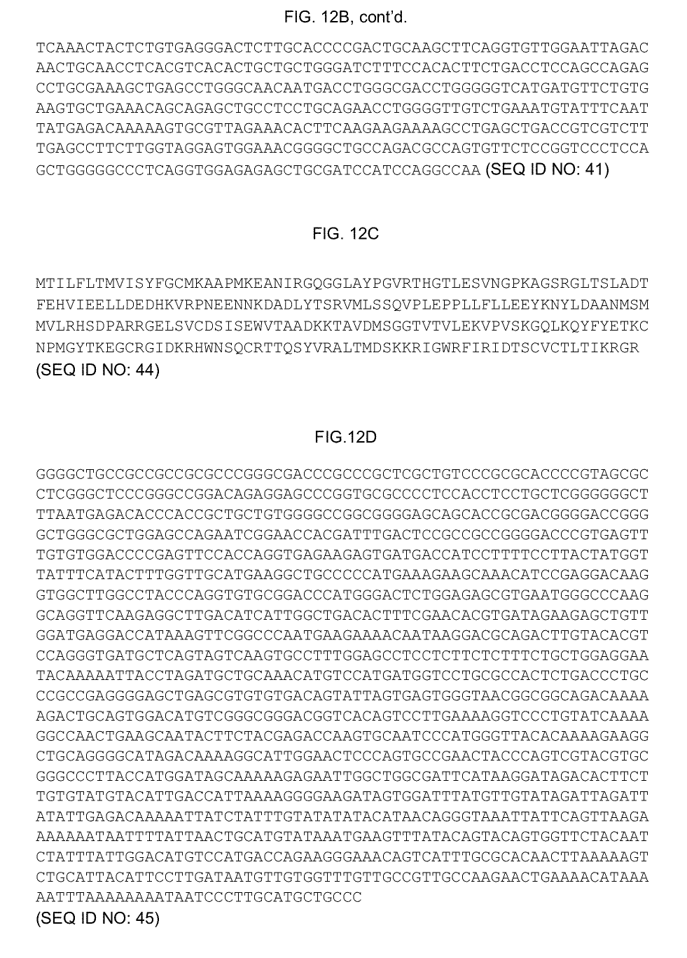

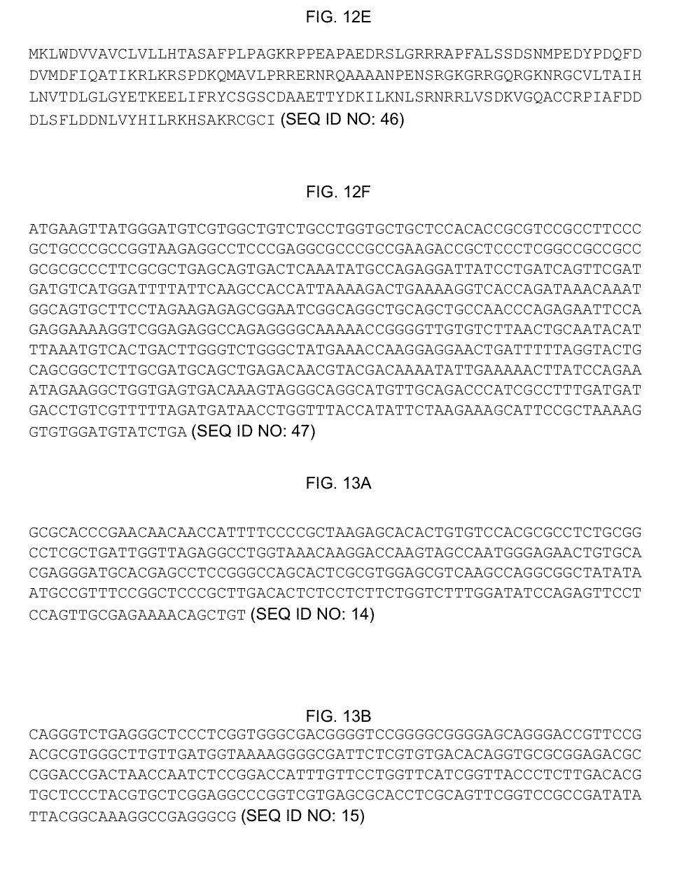

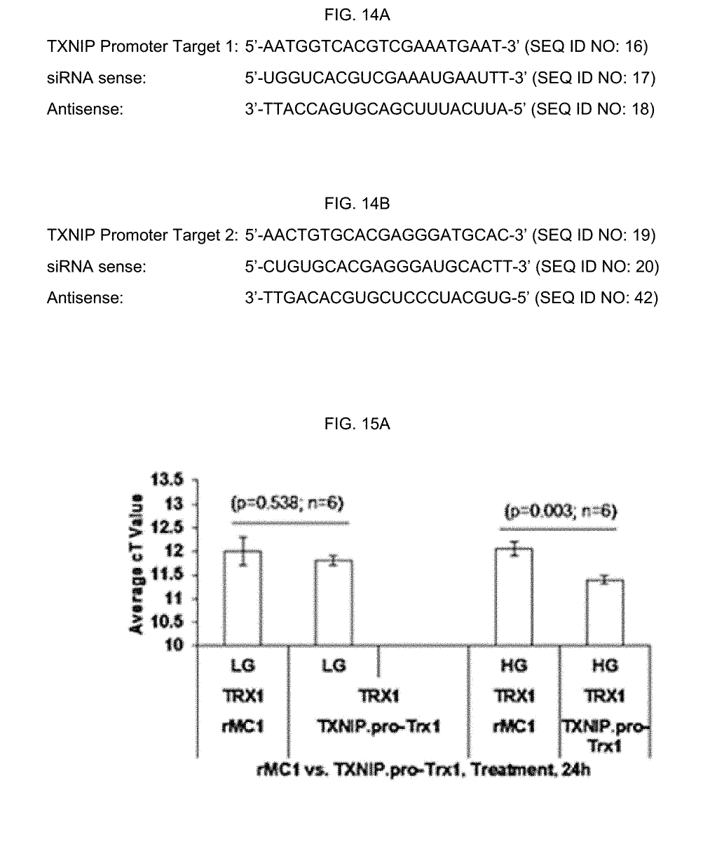

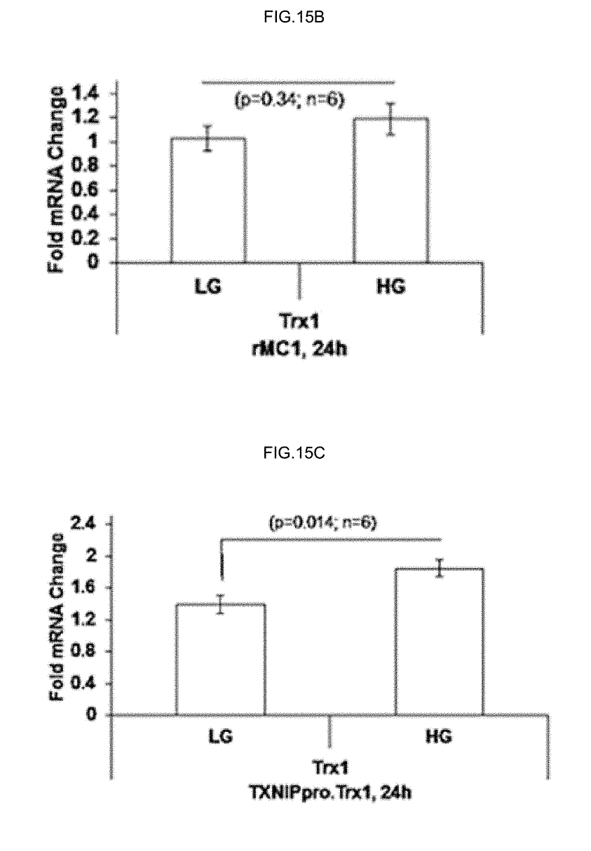

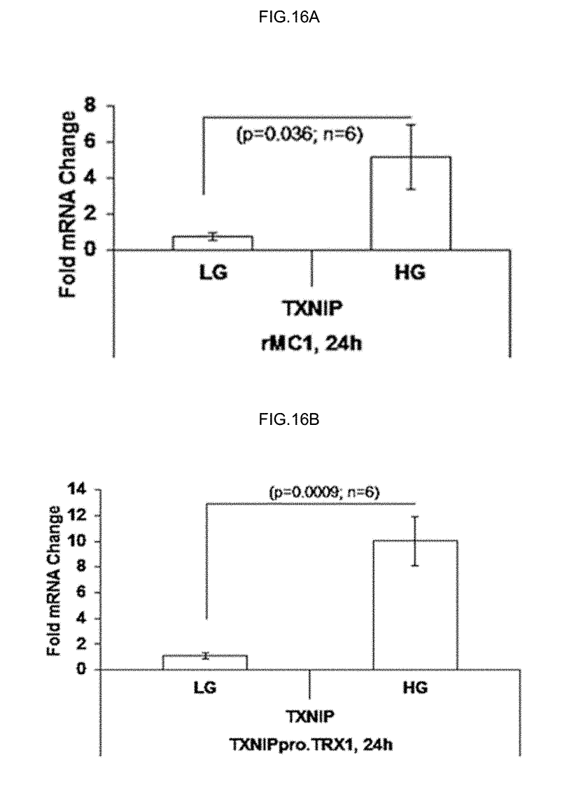

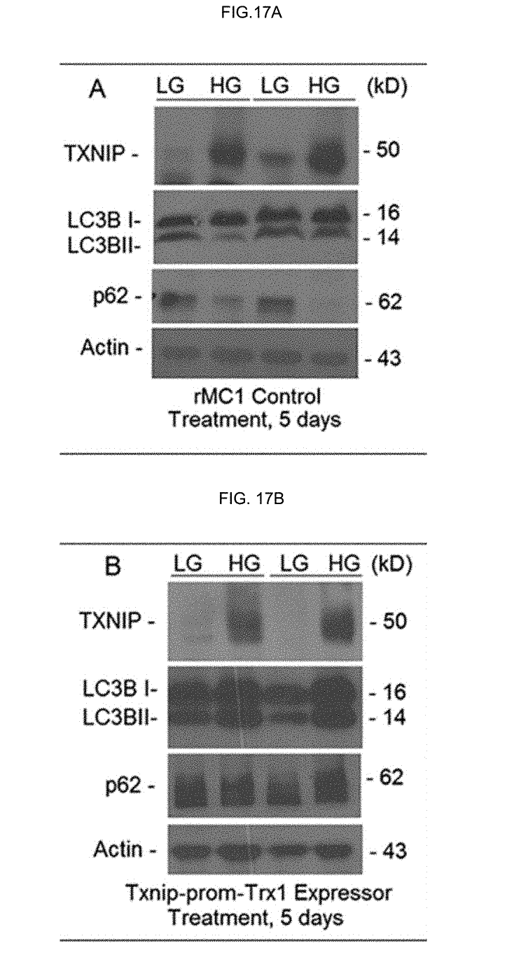

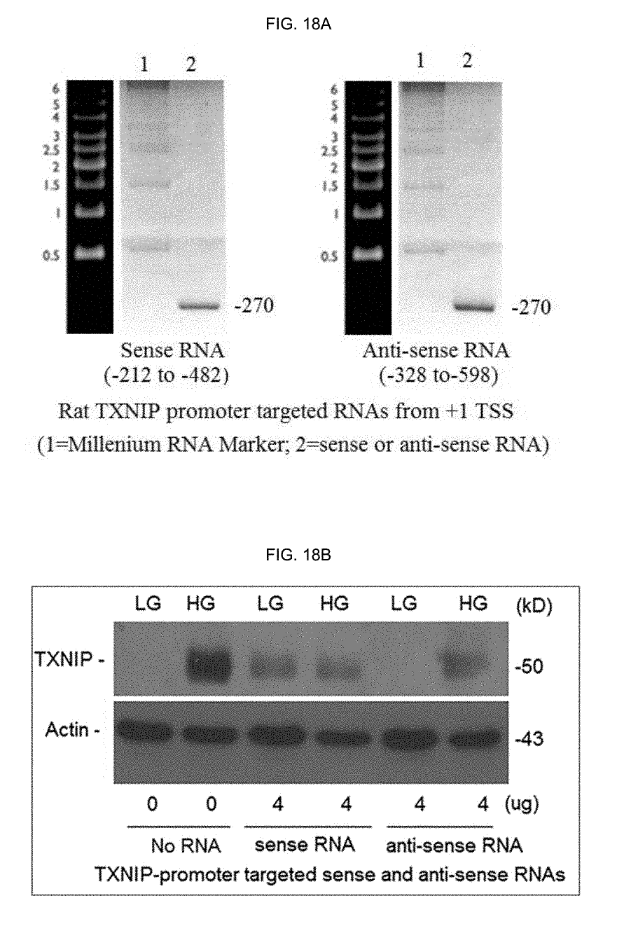

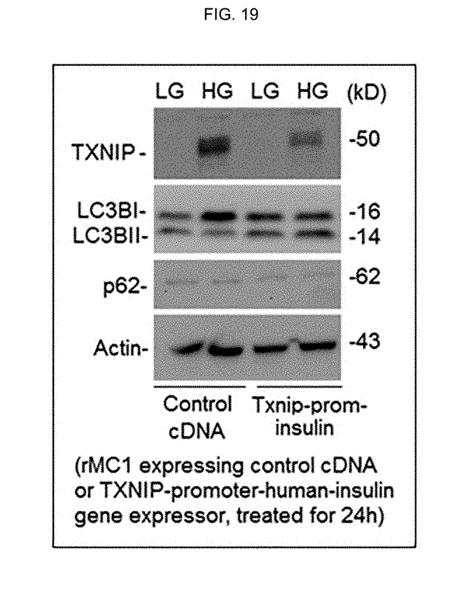

FIG. 10A provides an exemplary inducible nitric oxide synthase (iNOS) sequence (GenBank NP_000616.3, SEQ ID NO: 11). FIG. 10B provides an exemplary encoding cDNA sequence for iNOS that can be targeted for down-regulation (NM_000625.4; SEQ ID NO: 39).

FIG. 11A provides an exemplary hypoxia inducible factor 1-alpha (HIF-1alpha) sequence (GenBank NP_001521.1, SEQ ID NO: 12). FIG. 11B provides an exemplary encoding cDNA sequence for HIF-alpha that can be targeted for down-regulation (NM_001530.3; SEQ ID NO: 40).

FIG. 12A provides an exemplary NOD-like receptor family, pyrin domain containing 3 protein (NLRP3) sequence (GenBank AA143360.1, SEQ ID NO: 13). FIG. 12B provides an exemplary encoding cDNA sequence for NLRP3 that can be targeted for down-regulation (BC143359.1; SEQ ID NO: 41). FIGS. 12C and 12D provide exemplary Homo sapiens BDNF sequences (GenBank: X91251.1). FIG. 12C includes a protein translation (SEQ ID NO: 44) while FIG. 12D includes mRNA/cDNA (CDS 285..1028; SEQ ID NO: 45). FIGS. 12E and 12F provide exemplary Homo sapiens glial cell derived neurotrophic factor sequences (cDNA clone MGC:96936 IMAGE:7262145), complete cds; GenBank: BC069369.1). FIG. 12E includes a protein translation (SEQ ID NO: 46) while FIG. 12F includes mRNA/cDNA (CDS 1..636; SEQ ID NO: 47).

FIGS. 13A and 13B provide exemplary non-coding RNAs for gene silencing. The two non-coding RNAs are 270 nucleotides in length and target the sense and anti-sense sequence of an endogenous proximal TXNIP promoter (GI: 117514). FIG. 13A provides SEQ ID NO: 14 for targeting the antisense sequence of an endogenous proximal TXNIP promoter. FIG. 13B provides SEQ ID NO: 15 for targeting the sense sequence of an endogenous proximal TXNIP promoter.

FIGS. 14A and 14B provide exemplary iRNAs for targeting the TXNIP promoter. FIG. 14A provides: TXNIP Promoter Target 1 (SEQ ID NO: 16), iRNA sense (SEQ ID NO: 17), and antisense (SEQ ID NO: 18). FIG. 14B provides TXNIP Promoter Target 2 (SEQ ID NO: 19), iRNA sense (SEQ ID NO: 20), and antisense (SEQ ID NO; 42).

FIGS. 15A-15C show that the TXNIP promoter is activated by high glucose. Trx1 mRNA expression in control rMC1 cells is not significantly increased by high glucose (HG, 25 mM) compared to low glucose (LG, 5.5 mM) as indicated by cT values (FIG. 15A) and mRNA (FIG. 15B). However, stable transfection of the TXNIP.promoter Trx1 gene in rMC1 significantly increases message level as shown by a reduction in the cT value (FIG. 15A, right panel) and the fold change in Trx1 mRNA level (FIG. 15C) under HG showing that the TXNIP promoter is activated by HG.

FIGS. 16A and 16B show that TXNIP expression is increased by HG in both the control rMC1 (FIG. 16A) and TXNIP promoter Trx1 stably overexpressing rMC1 cells (FIG. 16B). This data shows that the TXNIP promoter is activated in both cell lines by HG.

FIGS. 17A and 17B show that TXNIP-prom-Trx1 inhibits TXNIP's effects on autophagy induction in rMC1 cells. (17A) High glucose induced TXNIP expression is associated with reducitons in autophagic double-membrane forming LC3BII protein and ubiquitin binding protein p62 indicating their flux to lysosomal degradation. (17B) Conversely, in TXNIP-prom-Trx1 rMC1 cells, high glucose still increases TXNIP expression, however, its downstream action on LC3BI and LC3BII as well as on p62 levels are increased, suggesting a blockade of the autophagic flux to lysosome and protein degradation. These results suggest that Trx1 nullifies the effect of TXNIP via its interaction as TXNIP is known to bind to Trx.

FIGS. 18A, 18B. FIG. 18A shows synthesis of the (left panel) sense and (right panel) antisense RNAs targeted to the TXNIP promoter. Both sense and anti-sense RNAs were synthesized by TriLink BioTechnologies (San Diego, Calif.). The sequences of these RNAs are shown in FIGS. 13A and 13B. Both RNAs show a single band corresponding to RNAs with 270 nt molecular weights indicating the purity of these synthetic products. FIG. 18B shows that the sense and anti-sense RNAs directed to the TXNIP promoter reduce TXNIP expression. Rat retinal rMC1 cells were transfected with 4 ug of sense or anti-sense RNA using Lipopfectamine 2000CD. These cells were then maintained in low glucose (5.5 mM) or high glucose (25 mM) for 3 days, then TXNIP protein levels were detected on Western blots. The results show that high glucose increases TXNIP expression in rMC1 cells in the absence of sense or anti-sense RNA transfection. On the other hand, transfection of sense and anti-sense targeted to TXNIP promoter reduces high glucose-induced TXNIP expression. Without being bound by theory, the mechanism(s) may include (i) inhibition of transcription factor binding to TXNIP promoter, (ii) epigenetic modification(s) at the TXNIP promoter, and/or (iii) formation of triple RNA-DNA complex at the promoter, which prevents transcription factor and co-factor binding.

FIG. 19 shows that TXNIP-prom-Insulin expression reduces TXNIP expression in rMC1. This plasmid was custom-prepared by Gene Script, Piscataway, N.J. In comparison to control pcNDA3.1 plasmid expression cells, Txnip-prom-insulin transfection in rMC1 reduces high glucose induced TXNIP expression. Similarly, some of the downstream effects of high glucose on LC3BII appear to be altered. Without being bound by theory the mechanism(s) may involve secretion of insulin into the culture media and its action on insulin receptors present in these cells whereby reducing TXNIP expression. Insulin and IGF-1 are known to inhibit the expression of TXNIP in various cells types under high glucose.

### DETAILED DESCRIPTION

Currently, diabetes mellitus (DM) afflicts over 240 million people worldwide. Type 1 diabetes accounts for 10% of the 240 million people, while type 2 diabetes accounts for the remaining 90% of the individuals.

Insulin is a peptide produced by beta cells in the pancreas. When there is high glucose in the blood, which occurs after a meal, beta cells secrete insulin into the blood. Insulin works to store excess blood glucose in liver, muscle, and fat as glycogen via its receptor at plasma membranes. When there is less glucose in the blood, then the stored glycogen can be broken down to free glucose by the action of glucagon. The glucose is used as fuel in the brain, eye, muscle and all other cell types to generate energy (ATP) mostly via mitochondrial oxidative phosphorylation in the electron transport chain.

The hyperglycemia observed in Type 1 diabetes results from a lack of insulin production due to an autoimmune mediated destruction of the beta cells of the pancreas. Patients require daily administration of insulin for survival and are at risk for ketoacidosis and other complications.

Type 2 diabetes results from insensitivity to insulin and/or a failure of the pancreatic beta-cells to keep up with the insulin requirements, resulting in hyperglycemia. Type 2 diabetes is primarily due to obesity and a lack of exercise. Type 2 DM may be treated with medications with or without insulin. Some of these treatments, however, can cause low blood sugar.

Gestational diabetes is a loss of blood sugar control that occurs during pregnancy and generally resolves after birth of the baby.

Diabetic retinopathy (DR) is one of the most severe complications of diabetes. It can cause poor vision and blindness. DR results from hyperglycemia induced changes of the vascular wall of the retinal blood vessels leading to the breakdown of the blood-retinal barrier making the retinal blood vessels more permeable. Blood and other liquids can leak into the retina causing blurry vision.

In the early stage, DR is known as non-proliferative DR (NPDR), and there are usually no symptoms associated with it or the symptoms are not visible to the eye. NPDR can only be detected by fundus photography. However, as the disease progresses, the NPDR enters the advanced stage and becomes proliferative DR (PDR), and new blood vessels grow or proliferate along the retina and in the vitreous humor that fills the inside of the eye. If left untreated, the new blood vessels can bleed, cloud the vision, and destroy the retina. The bleeding can also cause scar tissue to form which can pull on the retina and cause retinal detachment. The proliferation of the blood vessels can also cause neovascular glaucoma as the new blood vessels grow into the anterior chamber of the eye. Moreover, PDR can lead to macular edema, swelling of the middle of the retina, which can cause legal blindness. At present, DR is treated with laser surgery, injection of corticosteroids or anti-vascular endothelial growth factor (VEGF) into the eye, and vitrectomy. However, each of these treatment methods has disadvantages associated with it.

Insulin resistance occurs in Alzheimer's disease and is considered to contribute to the pathology. Therefore, Alzheimer's disease is also considered by some as Type 3 diabetes.

Under oxidative stress and inflammation, proteins aggregate (tau) and form plaques causing neurodegeneration. Diabetic retinopathy also causes neurodegeneration and is considered to be a window to the progression of Alzheimer's disease.

Dopamine neurons are injured in the retina in diabetic retinopathy and may contribute to eye movement defects and other neuro-visual signaling. Similarly, dopamine neurons are vulnerable to oxidative stress and aberrant protein accumulation (alpha-synuclein) and neurodegeneration, causing shaky motorneuron symptoms of the disease. However, all these neuronal diseases begin much earlier at the molecular level, and diabetes and aging-induced TXNIP overexpression, insulin resistance and oxidative stress play a causative role in neurodegeneration.

The current disclosure provides nucleic acid constructs including a thioredoxin-interacting protein (TXNIP) promoter operably linked to a gene of interest for treatment of a disease, such as the treatment of DM, DR, and age-related diseases such as Alzheimer's disease and Parkinson's disease.

TXNIP is a pro-oxidative stress, pro-inflammatory, and pro-apoptotic protein, strongly induced by high glucose and stress such as steroid hormones (e.g. glucocorticoids). TXNIP is a pro-diabetic and pro-apoptotic protein critically involved in the progression of diabetes and its complications. The TXNIP promoter and TXNIP's associated expression is upregulated by high glucose within minutes and is inhibited by insulin and IGF-1 in the cells of the retina and kidney, as well as cells of other tissues including the beta-cells of the pancreas and muscle cells. Therefore, in the absence of insulin, such as in Type 1 diabetes or in the case of insulin resistance, such as Type-2 diabetes, hyperglycemia persists and TXNIP upregulation is maintained.

TXNIP binds to thioredoxin (Trx), an anti-oxidant and redox regulating protein, and inhibits its activity, thereby causing cellular oxidative stress, inflammation, and apoptosis, which have been implicated in the onset and progression of DM. TXNIP silencing by iRNA (e.g., siRNA or shRNA) prevents several abnormalities or aberrant gene expressions in the diabetic rat retina and under high glucose conditions in retinal cells in culture.

In the diabetic rat retina and retina endothelial cells, TXNIP expression is regulated by histone acetylation, rather than by DNA methylation. Thus, the TXNIP promoter can be used to create nucleic acid constructs that enable therapeutic gene expression in high glucose conditions. As indicated, the TXNIP promoter can be operably linked to a gene encoding (i) insulin or an insulin-like or insulin-promoting protein; (ii) a protein that reduces cellular oxidative stress, inflammation and/or apoptosis; and/or (iii) an iRNA sequence that reduces expression of a protein that promotes cellular oxidative stress, inflammation and/or apoptosis. Each of these approaches can be used to treat DM and/or DR.

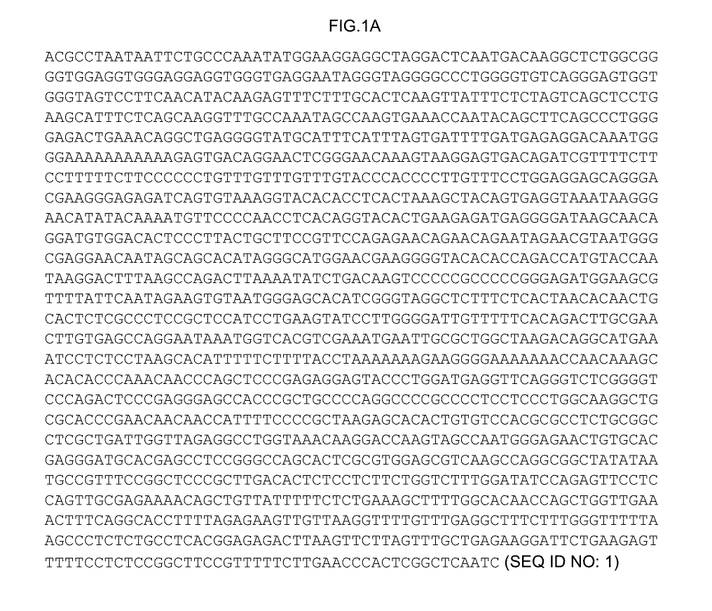

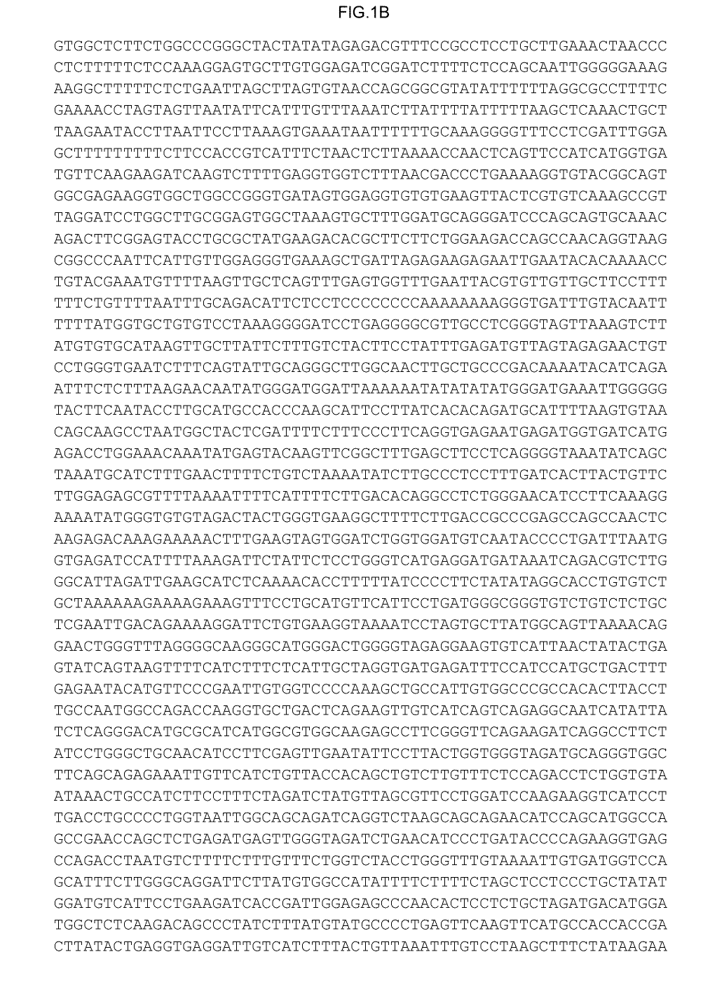

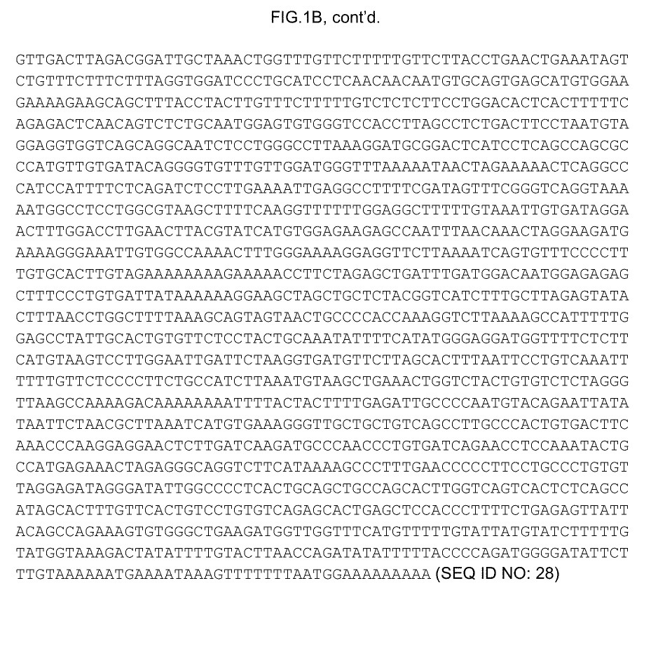

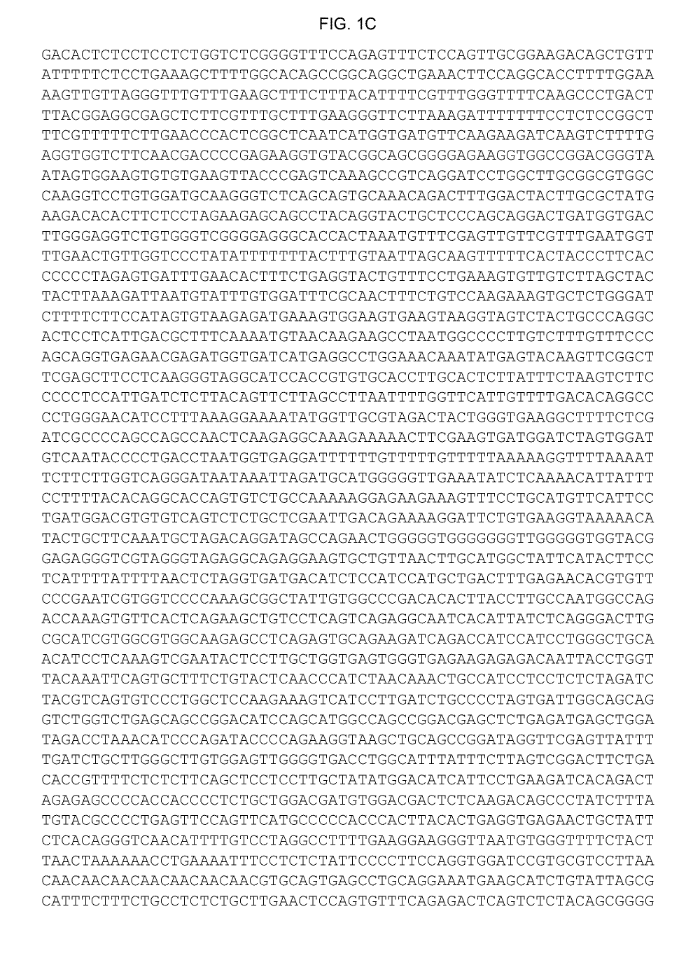

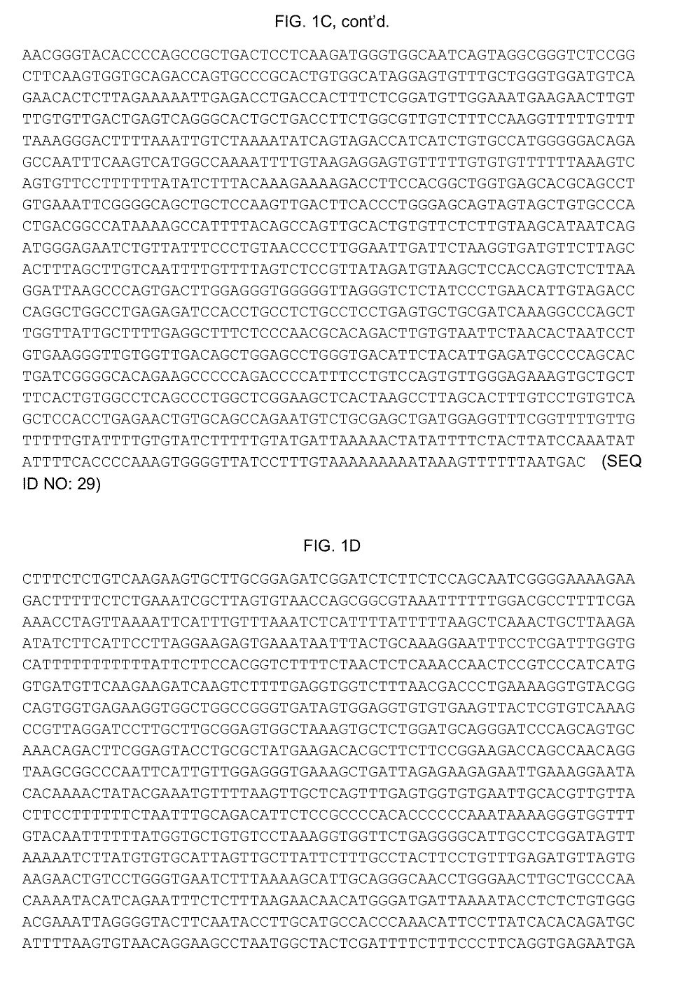

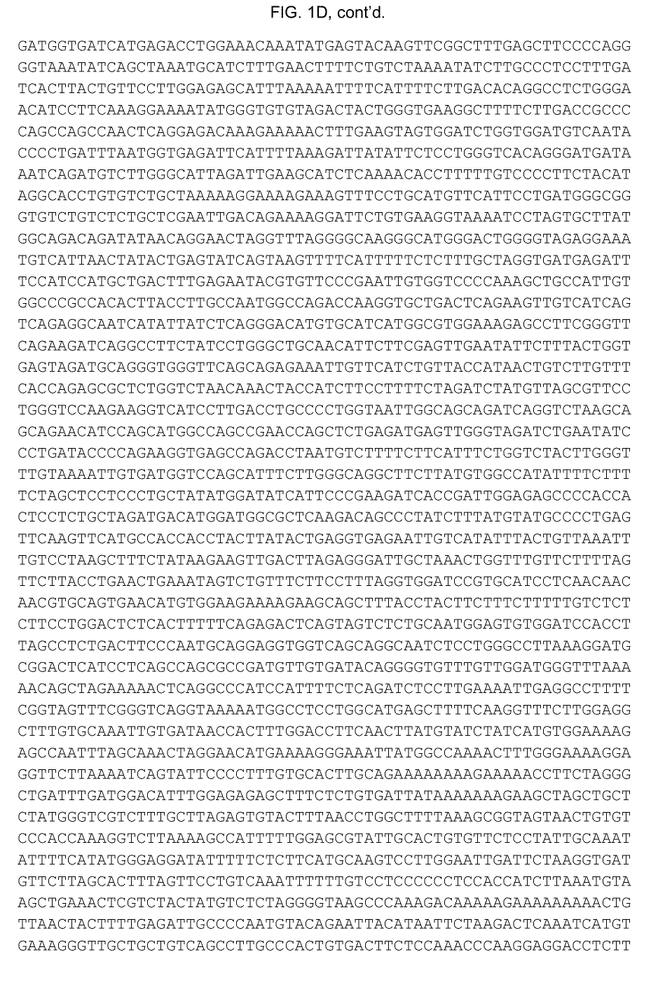

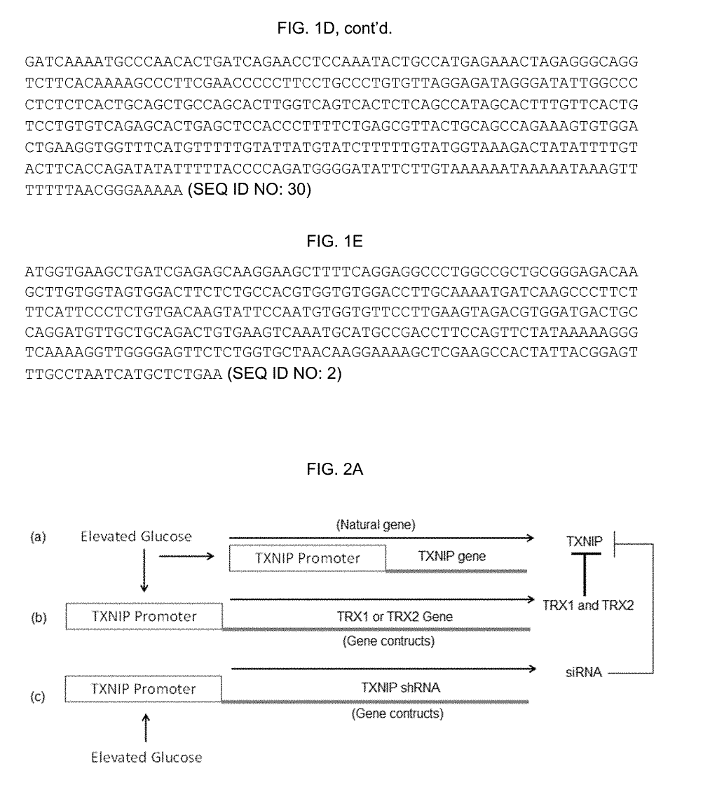

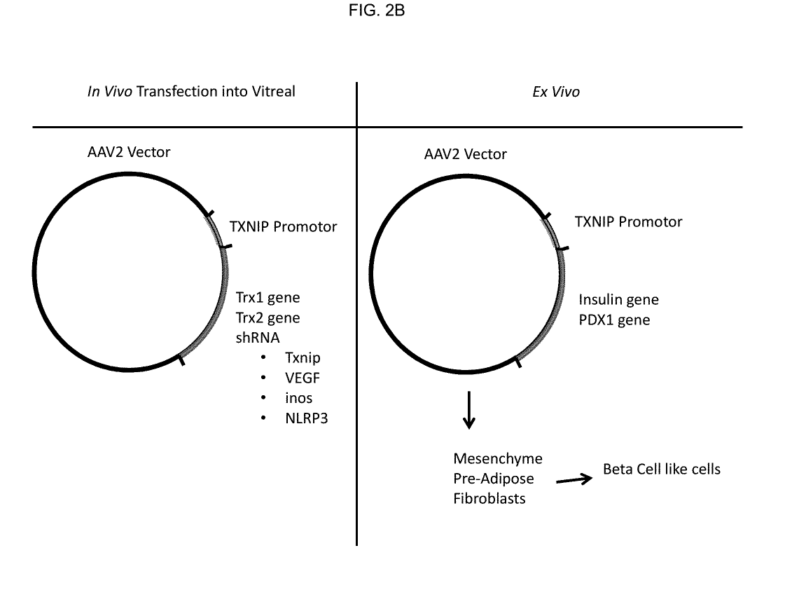

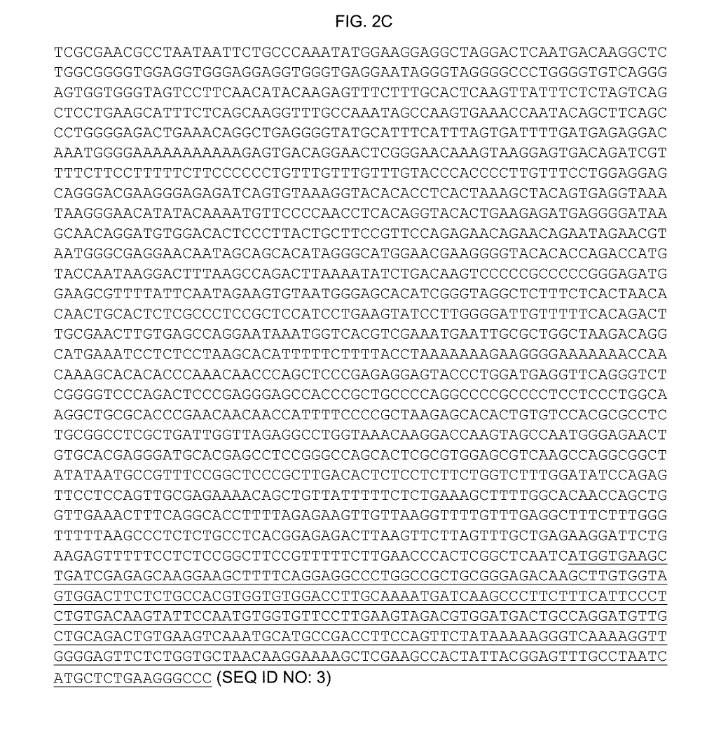

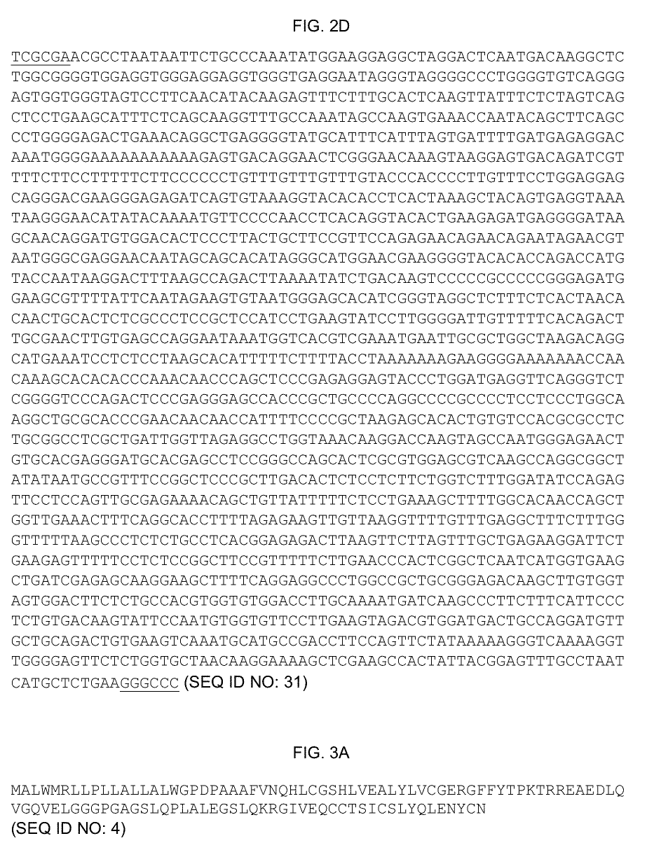

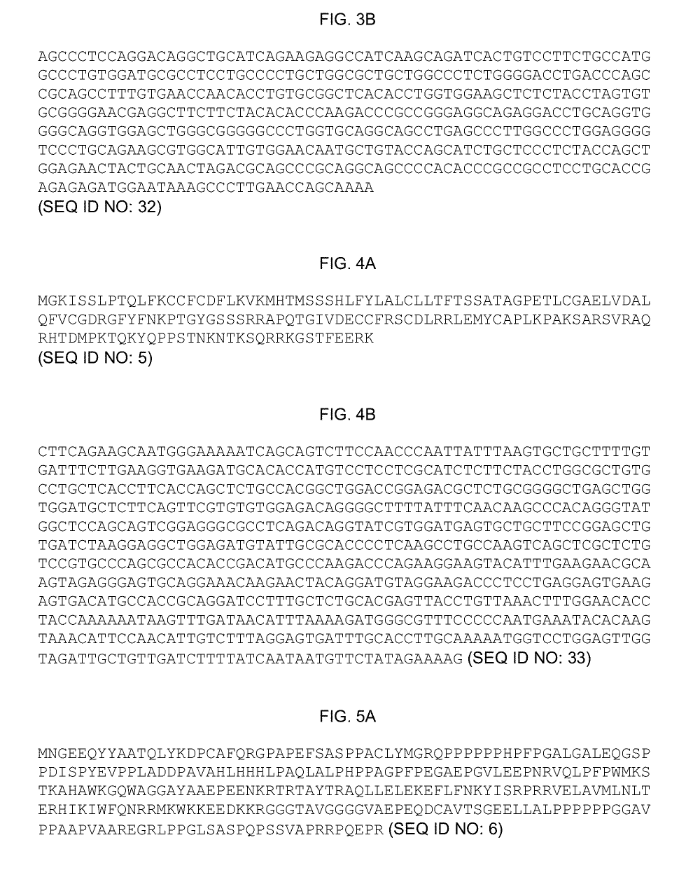

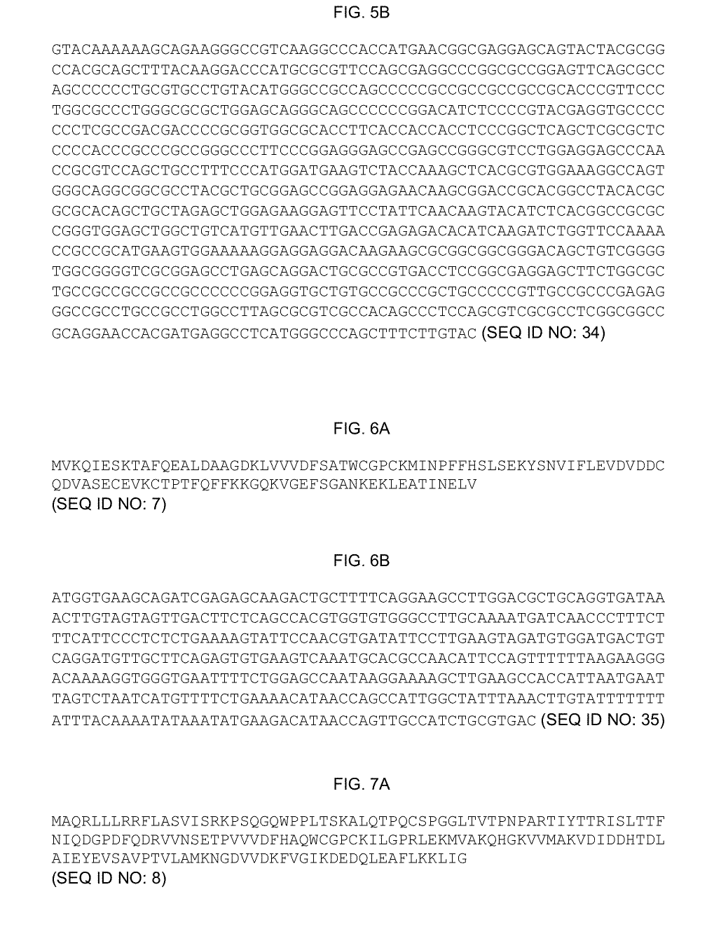

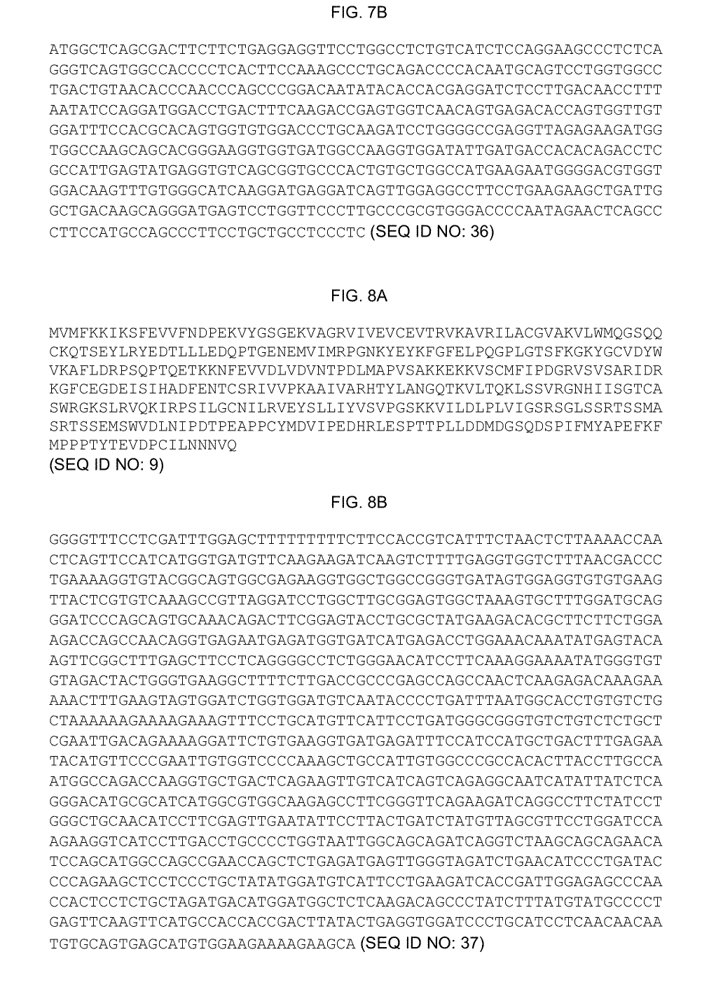

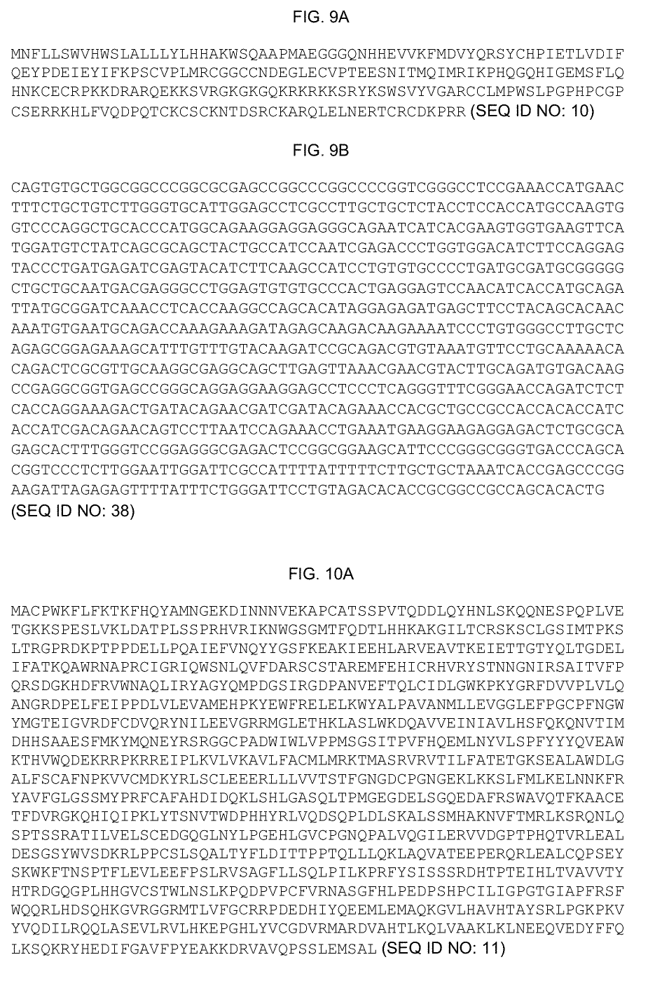

Exemplary relevant sequences for the TXNIP promoter can be found at FIG. 1A (a thioredoxin interacting protein (TXNIP) promoter region including nucleotides −1 to −1526 of Gene ID: 117514 (SEQ ID NO: 1)); FIG. 1B (a human TXNIP promoter found at gene ID: 10628 (SEQ ID NO: 28)); FIG. 10 (a mouse TXNIP promoter found at gene ID: 56338 (SEQ ID NO: 29)); and FIG. 1D (a rheus monkey TXNIP promoter found at gene ID 698683 (SEQ ID NO: 30)).

Insulin, Insulin-Like, and or Insulin-promoting Proteins. As explained previously, insulin is a peptide produced by beta cells in the pancreas. When there is high glucose in the blood, which occurs after a meal, beta cells secrete insulin into the blood. Insulin works to store excess blood glucose in liver, muscle, and fat as glycogen via its receptor at plasma membranes. When there is less glucose in the blood, then the stored glycogen can be broken down to free glucose by the action of glucagon. The glucose is used as fuel in the brain, eye, muscle and all other cell types to generate energy (ATP) mostly via mitochondrial oxidative phosphorylation in the electron transport chain. Exemplary relevant sequences for insulin can be found at Accession Nos. AAA59172.1, AAB60625.1, AAA19033.1, ACD35246.1, and P01315.2.

IGF-1 is a hormone similar in molecular structure to insulin (e.g., insulin-like). It plays an important role in childhood growth and continues to have anabolic effects in adults. Binding of IGF-1 to its receptor (IGF1R), a receptor tyrosine kinase, initiates intracellular signaling. IGF-1 is one of the most potent natural activators of the AKT signaling pathway, a stimulator of cell growth and proliferation, and a potent inhibitor of programmed cell death. Exemplary relevant sequences for IGF-1 can be found at Accession Nos. NP_001104753.1, NP_001071296.1, and NP_001004384.1.

PDX1 activates insulin (e.g., insulin-promoting), somatostatin, glucokinase, islet amyloid polypeptide and glucose transporter type 2 gene transcription. In particular, PDX1 is involved in glucose-dependent regulation of insulin gene transcription. Exemplary relevant sequences for PDX1 can be found at Accession Nos. NP_000200, NP_001074947, and A1YF08.

Proteins that Reduce Cellular Oxidative Stress, Inflammation and/or Apoptosis.

Examples of proteins that reduce cellular oxidative stress, inflammation and/or apoptosis include the thioredoxins. Thioredoxins are proteins that act as antioxidants by facilitating the reduction of other proteins by cysteine thiol-disulfide exchange. Thioredoxins are found in nearly all known organisms and are essential for life in mammals. Examples of thioredoxins include thioredoxin 1 (Trx1) and thioredoxin 2 (Trx2). Trx1 is expressed in the cell nucleus and cytosol, while Trx2 is expressed in cell mitochondria. Exemplary relevant sequences for Trx1 can be found at Accession Nos. AAF86466.1, NP_446252.1, and NP_037950.1. Exemplary relevant sequences for Trx2 can be found at Accession Nos. AAF86467.1 and NP_064297.1.

iRNA Sequences that Reduce Expression of a Protein that Promotes Cellular Oxidative Stress, Inflammation and/or Apoptosis.

The current disclosure also describes nucleic acid constructs encoding iRNA that can be used to reduce expression of proteins that promote cellular oxidative stress, inflammation and/or apoptosis. Exemplary proteins include TXNIP, Vascular Endothelial Growth Factor (VEGF), inducible nitric oxide synthases (iNOS), hypoxia-inducible factor 1-alpha (HIF-1alpha), and NOD-like receptor family, pyrin domain containing 3 protein (NLRP3).

As stated, TXNIP is a pro-diabetic and pro-apoptotic protein involved in diabetes and its complications that binds and inhibits the activity of Trx. Exemplary relevant sequences for TXNIP can be found at Accession Nos. AAH93704.1, NP_001008767.1, and AAH11212.1.

iRNA sequences that reduce TXNIP expression include those that target an endogenous TXNIP promoter region (e.g., one that is not part of a nucleic acid construct in operable combination with a therapeutic gene as provided herein). As an example, the two non-coding sequences shown in FIGS. 13A and 13B target the sense and anti-sense sequences of the endogenous proximal TXNIP promoter. As another example, the siRNAs shown in FIGS. 14A and 14B target the endogenous TXNIP promoter.

VEGF is a protein produced by cells that stimulate vasculogenesis and angiogenesis. It is part of the system that restores the oxygen supply to tissues when blood circulation is inadequate. Serum concentration of VEGF is high in bronchial asthma and diabetes mellitus. Overexpression of VEGF can cause vascular disease in the retina and other parts of the body. VEGF is also implicated in the neovascularization of PDR specifically, as well as angiogenesis of islets in the pancreatic developmental stage in determining beta cell mass and properties. Examples of VEGFs include VEGF-A, VEGF-B, VEGF-C, VEGF-D, VEGF-D, and PIGF (placental inhibitory growth factor). As a result of alternative splicing of mRNA from a single, 8-exon VEGF-A gene, there exist multiple forms of VEGF-A. Exemplary relevant sequences for VEGF-A can be found at Accession Nos. P15692.2, NP_001020281.1, NP_001003175.2, NP_001103972.1, NP_001303972.1, NP_001274043.1, and AAH61468.1.

Nitric oxide (NO) plays an important role in modulating vascular tone, insulin secretion, and peristalsis, and is involved in angiogenesis and neural development. The production of NO from L-arginine is catalyzed by a family of enzymes known as nitric oxide synthases (NOSs). There are three isoforms of NOS that mediate NO production: eNOS (endothelial NOS), nNOS (neuronal NOS), and iNOS. iNOS is synthesized by various cell types in response to cytokines. iNOS produces large amounts of NO as a defense mechanism, such as in the response of the body to attack by parasites, bacterial infection, and tumor growth. iNOS is also the cause of septic shock. Oxidative stress induces iNOS expression and NO synthesis. Moreover, it has been shown that islet iNOS expression is induced resulting in high NO concentration in acute pancreatitis, and type 1 and type 2 diabetes mellitus. Exemplary relevant sequences for iNOS can be found at Accession Nos. NP_000616.3, NP_001300851.1, NP_001300851.1, and NP_036743.3.

A subunit of a heterodimeric transcription factor hypoxia-inducible factor 1 (HIF-1) is considered as the master transcriptional regulator of cellular and developmental response to hypoxia. The dysregulation and overexpression of HIF-1alpha by either hypoxia or genetic alterations have been heavily implicated in cancer biology, as well as a number of other pathophysiologies, specifically in areas of vascularization and angiogenesis, energy metabolism and cell survival. HIF-1alpha mediates the transcriptional activation of VEGF. Exemplary relevant sequences for HIF-1alpha can be found at Accession Nos. NP_001521.1, NP_851397.1, NP_001230013.1, NP_077335.1, and AAH26139.1.

The NLR family, pyrin domain containing 3 gene (NLRP3 gene) encodes the NLRP3 protein. NLRP3 belongs to the family of proteins called nucleotide-binding domain and leucine-rich repeat containing (NLR) proteins. When activated, NLRP3 proteins assemble themselves along with other proteins into inflammasomes, which mediate the process of inflammation. The aberrant activation of NLRP3 is associated with various disorders including diabetes, particularly type 2 diabetes. Exemplary relevant sequences for NLRP3 can be found at Accession Nos. AA143360.1, NP_001178571.1, and AA116176.1.

As indicated, nucleic acid constructs disclosed herein include at least a TXNIP promoter operably linked to a gene encoding a therapeutic protein or an iRNA. The nucleic acid construct can be used for nucleic acid expression including transcription and translation of the gene operably linked to the promoter in the construct. The nucleic acid construct can also be used to replicate the gene included in the construct. In addition to the promoter, the nucleic acid construct can include other regulatory elements, such as a terminator, a poly-A sequence, an origin of replication, and a ribosomal binding sequence.

The term “promoter” refers to at least a region of the DNA that is involved in recognition and binding of RNA polymerase and other proteins to initiate transcription of DNA that is operably linked to it. The term “promoter” includes the full length promoter or a portion of the full length promoter sufficient for binding RNA polymerase and other proteins to initiate transcription. Additionally, the promoter can include sequences that modulate the binding and transcription initiation activity of the RNA polymerase, such as the cis acting or the trans acting factors. An example of a promoter region described in the current disclosure is nucleotides −1 to −1526 of the TXNIP gene (GI: 117154) shown in FIG. 1A.

The term “operably linked” refers to a first sequence located sufficiently close to a second sequence such that the first sequence can influence or control the second sequence. As an example, a promoter sequence can be operably linked to a gene sequence, and is normally located at the 5′-terminus of the gene sequence such that the expression of the gene sequence is under the control of the promoter sequence. Additionally, one or more regulatory sequences are operably linked to a promoter sequence in order to enhance the ability of the promoter sequence in promoting transcription. The regulatory sequence is generally located at the 5′-terminus of the promoter sequence.

The term “gene” refers to a nucleic acid sequence that encodes one or more therapeutic proteins or iRNA sequences as described herein. This definition includes various sequence polymorphisms, mutations, and/or sequence variants wherein such alterations do not substantially affect the function of the encoded therapeutic proteins or iRNA. The term “gene” may include not only coding sequences but also regulatory regions such as promoters, enhancers, and termination regions. The term further can include all introns and other DNA sequences spliced from the mRNA transcript, along with variants resulting from alternative splice sites. Gene sequences encoding the molecule can be DNA or RNA that directs the expression of the one or more therapeutic proteins. These nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein or iRNA. The sequences can also include degenerate codons of the native sequence or sequences that may be introduced to provide codon preference in a specific cell type.

A gene sequence encoding one or more therapeutic proteins and/or iRNA sequences can be readily prepared by synthetic or recombinant methods from the relevant amino acid sequence. In particular embodiments, the gene sequence encoding any of these sequences can also have one or more restriction enzyme sites at the 5′ and/or 3′ ends of the coding sequence in order to provide for easy excision and replacement of the gene sequence encoding the sequence with another gene sequence encoding a different sequence. In particular embodiments, the gene sequence encoding the sequences can be codon optimized for expression in mammalian cells.

In particular embodiments, the encoded therapeutic genes and/or iRNA-targeted genes include those that have at least 90%, 91%, 92%, 93%, 94%, 95%, 96%, 97%, 98%, or 99% sequence identity to insulin, IGF-1, PDX1, Trx1, Trx2, TXNIP, VEGF-A, iNOS, HIF-1alpha, or NLRP3. In particular embodiments, the therapeutic genes and/or iRNA-targeted genes include those that encode a protein having at least 90%, 91%, 92%, 93%, 94%, 95%, 96%, 97%, 98%, or 99% sequence identity to insulin, IGF-1, PDX1, Trx1, Trx2, TXNIP, VEGF-A, iNOS, HIF-1alpha, or NLRP3.

“% sequence identity” refers to a relationship between two or more sequences, as determined by comparing the sequences. In the art, “identity” also means the degree of sequence relatedness between sequences as determined by the match between strings of such sequences. “Identity” (often referred to as “similarity”) can be readily calculated by known methods, including those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine sequence identity are designed to give the best match between the sequences tested. Methods to determine sequence identity and similarity are codified in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul, et al., J. Mol. Biol. 215, 403-410 (1990); DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y.). Within the context of this disclosure it will be understood that where sequence analysis software is used for analysis, the results of the analysis are based on the “default values” of the program referenced. “Default values” mean any set of values or parameters which originally load with the software when first initialized.

Reference to proteins described herein also include variants, modifications, D-substituted analogs, homologues and allelic variants thereof. “Variants” of proteins disclosed herein include proteins having one or more amino acid additions, deletions, stop positions, or substitutions, as compared to a protein disclosed herein.

An amino acid substitution can be a conservative or a non-conservative substitution. Variants of proteins disclosed herein can include those having one or more conservative amino acid substitutions. A “conservative substitution” involves a substitution found in one of the following conservative substitutions groups: Group 1: alanine (Ala or A), glycine (Gly or G), Ser, Thr; Group 2: aspartic acid (Asp or D), Glu; Group 3: asparagine (Asn or N), glutamine (Gln or Q); Group 4: Arg, lysine (Lys or K), histidine (His or H); Group 5: Ile, leucine (Leu or L), methionine (Met or M), valine (Val or V); and Group 6: Phe, Tyr, Trp.

Additionally, amino acids can be grouped into conservative substitution groups by similar function, chemical structure, or composition (e.g., acidic, basic, aliphatic, aromatic, sulfur-containing). For example, an aliphatic grouping may include, for purposes of substitution, Gly, Ala, Val, Leu, and Ile. Other groups containing amino acids that are considered conservative substitutions for one another include: sulfur-containing: Met and Cys; acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information is found in Creighton (1984) Proteins, W.H. Freeman and Company.

Induced or increased expression of a therapeutic protein is relative to a comparative expression level in a control cell that does not include a nucleic acid construct with a TXNIP promoter in operable combination with the therapeutic gene as disclosed herein. Induced or increased expression includes up-regulated expression of at least 10%, 20%, 30%, 40%, 50%, 60%, 70%, 80%, 90%, or 100% relative to its comparative expression level in a control cell. Methods to determine protein expression levels are well known in the art and include, for example, enzyme-linked immunosorbent assay (ELISA) and Western blotting. Increased expression can also be seen by a detectable change in a cell or a subject as compared with a control cell or subject (e.g., by a functional or symptom-based assay).

As indicated, in particular embodiments, the expression of targeted genes and proteins is reduced by RNA interference. Interfering RNA (iRNA) includes any type of RNA molecule capable of down-regulating expression of a target gene or protein including antisense RNA, short interfering RNA (siRNA), microRNA (miRNA), double-stranded RNA (dsRNA), hairpin RNA (hRNA, including short hRNA (shRNA)), sense RNA, ribozyme, and the like.

MicroRNA are genomically encoded non-coding RNAs that regulate gene expression by directing their target mRNAs for degradation or translational repression. Mature miRNAs are structurally similar to short interfering RNAs (siRNA), derived from cleavage of exogenous or foreign dsRNA. However, miRNAs differ from siRNAs in that miRNAs, especially those in animals, have incomplete base pairing to a target and inhibit translation of many different mRNAs with similar sequences, while siRNAs base-pair perfectly and induce mRNA cleavage only at a specific target.

In particular embodiments, the iRNA molecule has a length of at least 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 220, 240, 250, 260, 270, 280, 300, 400, 500, or 600 nucleotides.

Reduced expression can be used interchangeably with “suppressing” or “inhibiting” expression of a target gene and its encoded protein. Reduced expression is relative to a comparative expression level in a control cell that does not express iRNA encoded by a TXNIP promoter nucleic acid construct disclosed herein. Silencing includes down-regulation of transcription and accumulation of the RNA transcript encoded by the target gene and/or translation of the target gene into protein. Reduced expression includes a situation in which the expression level of the iRNA-targeted gene is reduced by 10%, 20%, 30%, 40%, 50%, 60%, 70%, 80%, 90%, or 100% relative to its comparative expression level in a control cell. Down-regulation also includes a situation in which encoded protein of the iRNA-targeted gene is decreased by 10%, 20%, 30%, 40%, 50%, 60%, 70%, 80%, 90%, or 100% relative to its comparative expression level in a control cell. Reduced expression by iRNA can also be seen by a detectable change in a cell or a subject as compared with a control cell or control subject.

Methods to assay for functional iRNA molecules are well known in the art. The methods include detecting reductions in RNA or protein levels which include RNA solution hybridization, Northern hybridization, reverse transcription (e.g. quantitative RT-PCR analysis), microarray analysis, antibody binding, enzyme-linked immunosorbent assay (ELISA) and Western blotting.

The nucleic acids described herein can be introduced into cells by techniques known in the art. The term “introducing a nucleic acid into a cell” includes any method for introducing an exogenous nucleic acid molecule into a selected host cell including transformation, transfection and transducing. Examples of such methods include calcium phosphate- or calcium chloride-mediated transfection, electroporation, microinjection, particle bombardment, liposome-mediated transfection, transfection using bacterial bacteriaphages, transduction using retroviruses or other viruses (such as vaccinia virus or baculovirus of insect cells), cell fusion, chromosome-mediated gene transfer, microcell-mediated gene transfer, sheroplast fusion, cell penetrating peptides, or other methods.

The liposome method is an approach using liposomes such as cationic liposomes, for example, cholesterol-based cationic liposomes. The method of using liposomes also includes lipofection, which utilizes the anionic electric properties of the cell surface. Alternatively, liposomes having surface bound with a cell membrane-permeable peptide (e.g., HIV-1 Tat peptide, penetratin, and oligoarginine peptide) can be used.

In particular embodiments, the nucleic acids described herein are stably integrated into the genome of a host cell. In particular embodiments, the nucleic acids are stably maintained in a cell as a separate, episomal segment. Transposons and transposable elements can be used to improve the efficiency of integration, the size of the DNA sequence integrated, and the number of copies of a DNA sequence integrated into a genome. Transposons or transposable elements include a short nucleic acid sequence with terminal repeat sequences upstream and downstream. Active transposons can encode enzymes that facilitate the excision and insertion of nucleic acid into a target DNA sequence. Examples of transposable elements that facilitate insertion of nucleic acids into the genome of mammals include sleeping beauty (e.g., derived from the genome of salmonid fish); piggyback (e.g., derived from lepidopteran cells and/or the Myotis lucifugus); mariner (e.g., derived from Drosophila); frog prince (e.g., derived from Rana pipiens); Tol2 (e.g., derived from medaka fish); TcBuster (e.g., derived from the red flour beetle Tribolium castaneum) and spinON.

In particular embodiments, the nucleic acids can incorporate chemical groups that alter the physical characteristics of the nucleic acid and retard degradation in the target cell. As an example, the internucleotide phosphate ester can be optionally substituted with sulfur.

In particular embodiments, nucleic acid constructs can be delivered using cell penetrating peptides. CPPs are short peptides that facilitate cellular uptake of various molecular cargo (from nanosize particles to small chemical molecules and large fragments of DNA). The “cargo” is associated with the peptides either through chemical linkage via covalent bonds or through non-covalent interactions. CPPs are of different sizes, amino acid sequences, and charges but all CPPs have one distinct characteristic: the ability to translocate the plasma membrane and facilitate the delivery of various molecular cargoes intracellularly. CPPs may enter cells through, for example, direct penetration of the membrane, endocytosis-mediated entry, or translocation through the formation of a transitory structure. Examples of CPPs include a transportan peptide (TP; e.g., GWTLNSAGYLLGKINLKALAALAKKIL (SEQ ID NO: 21)), a TP10 peptide (e.g., AGYLLGKINLKALAALAKKIL (SEQ ID NO: 22)), a pVEC peptide (e.g., LLIILRRRIRKQAHAHSK (SEQ ID NO: 23)), a penetratin peptide (e.g., RQIKIWFQNRRMKWKK (SEQ ID NO: 24)), a tat fragment peptide (e.g., GRKKRRQRRRPPQC (SEQ ID NO: 25)), a signal sequence based peptide (e.g., GALFLGWLGAAGSTMGAW (SEQ ID NO: 26)), and an amphiphilic model peptide (e.g., KLALKLALKALKAALKLA (SEQ ID NO: 27)).

The current disclosure also provides vectors including the nucleic acid constructs described herein. A vector is a vehicle for transporting a foreign genetic material, for example into another cell to be replicated or expressed. The vector can be an expression vector for expressing the protein encoded by the nucleic acid in the vector or a transcription vector for amplifying the nucleic acid.

Vectors include viruses, phages, a DNA vector, a RNA vector, a viral vector, a bacterial vector, a plasmid vector, a cosmid vector, and an artificial chromosome. The plasmids are plasmids for animal cells, such as plasmids for mammals. The plasmid vectors can belong to the pBluescript series or the pUC series. Artificial chromosomes include BAC and PAC.

Examples of viruses include adenovirus, adeno-associated virus, retrovirus, pox virus, herpes simplex virus (HSV), and hemagglutinating virus of Japan. Adenoviruses include Ad3, Ad5, Ad7, Ad11, and Ad3/5 chimera. Retroviruses include gammaretroviruses lentiviruses and foamy viruses. Gammaretroviruses include mouse stem cell virus, murine leukemia virus, feline leukemia virus, feline, sarcoma virus, and avian reticuloendotheliosis virus. Lentiviruses include human immunodeficiency virus (HIV) such as HIV type 1 and type 2); equine infectious anemia virus; feline immunodeficiency virus (FIV); bovine immune deficiency virus (BIV); and simian immunodeficiency virus (SIV). Foamy viruses include human foamy virus, simian foamy virus, and feline foamy virus. Pox virus includes vaccinia virus. Herpes simplex viruses include HSV-1 and HSV-2.

Retroviral vectors include those based on murine leukemia virus, gibbon ape leukemia virus (GALV), SIV, HIV, and combinations thereof.

Additionally, viral vectors can be derived adeno-associated viruses (AAV); alphaviruses; cytomegaloviruses (CMV); flaviviruses; influenza viruses; and papilloma viruses such as human and bovine papilloma viruses. Examples of viral vectors include a modified vaccinia Ankara (MVA) and NYVAC, or strains derived therefrom; avipox vector, such as fowl pox vector (FP9); or canarypox vectors (e.g., ALVAC and strains derived therefrom).

The vectors described herein can further include regulatory sequences such as a terminator, a poly-A sequence, a ribosomal binding sequence, a selective marker sequence, a reporter gene, an antibiotic-resistance gene, an enhancer sequence.

The current disclosure also includes cells genetically modified to express a nucleic acid construct disclosed herein. In particular embodiments, the cell is a genetically modified cell for use in a genetic therapy. In particular embodiments, the cell is a research or manufacturing cell. Exemplary genetically-modified cell types can include human cells, subject cells, embryonic cells, embryonic stem cells, tissue stem cells, fetal cells, epithelial cells, fibroblast cells, neural cells, keratinocytes, hematopoietic cells, epidermal cells, endothelial cells, beta-cells, non-beta cells, mesenchymal cells, adipose stem cells, pre-adipocytes, adipocytes, and muscle cells, cells obtained from a variety of different organs and tissues (e.g., skin, lung, pancreas, heart, intestine, stomach, bladder, blood vessels, kidney, urethra, or reproductive organs), mammalian cells (e.g., primate cells, monkey cells, murine cells, porcine cells, bovine cells, ovine cells, rodent cells, hamster cells, HEK293 cells, CHO cells, BHK cells, COS cells, HeLa cells, and MC1 cells), prokaryotic cells, eukaryotic cells, bacterial cells, E. coli., insect cells, plant cells, yeast, cultured cells, primary cultured cells, subcultured cells, established cell lines, transformed cells, transfected cells, somatic cells, germ cells, etc.

Moreover, the current disclosure describes compositions including the disclosed nucleic acid constructs and a carrier. In particular embodiments, the carrier is a pharmaceutically acceptable carrier.

Injectable compositions can include one or more nucleic acid constructs disclosed herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, or solutes.

As an example, injectable compositions can be formulated as aqueous solutions, such as in buffers including Hanks' solution, Ringer's solution, or physiological saline. The aqueous solutions can contain formulatory agents such as suspending, stabilizing, and/or dispersing agents. Examples of suitable aqueous and non-aqueous carriers, which may be employed in the injectable formulations include water, ethanol, polyols (such as glycerol, propylene glycol, polyethylene glycol, and the like), and suitable mixtures thereof, vegetable oils, such as olive oil, and injectable organic esters, such as ethyloleate. Proper fluidity can be maintained, for example, by the use of coating materials, such as lecithin, by the maintenance of selected particle size in the case of dispersions, and by the use of surfactants.

Injectable formulations can also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like in the compositions. In addition, prolonged absorption of the injectable pharmaceutical form may be brought about by the inclusion of agents which delay absorption such as aluminum monostearate and gelatin.

Alternatively, the composition can be in lyophilized form and/or provided in powder form for constitution with a suitable vehicle, e.g., sterile pyrogen-free water, before use. Lyophilized compositions can include less than 5% water content; less than 4.0% water content; or less than 3.5% water content.

The composition can be in a unit dosage form, such as in a suitable diluent in sterile, hermetically sealed ampules or sterile syringes.

In particular embodiments, a carrier can also include a genetically-modified cell, as explained below. Suitable carriers and diluents for cells include isotonic saline solutions, for example phosphate-buffered saline. Cell-containing compositions typically are formulated for intravenous or subcutaneous administration, or for administration by transplantation.

In particular embodiments, the cells are encapsulated. Generally the encapsulating material is permeable to nutrients (such as sugars or amino acids), but impermeable to immune mediators (such as antibodies or complement components) or cell. The material can include alginate (alternating blocks of mannuronic and gluronic acid) such as in the form of barium and/or poly-L-lysine alginate. The material can include hollow fibers (such as acrylic, polyacrylonitrile vinyl chloride or polyethersulfone). The material can include hydroxyethyl-methacrylate-methyl-methacrylate, polyphosphazene or agarose.

Injectable ophthalmic formulations can be prepared as solutions, suspensions, ointments, gels, emulsions, oils, and other dosage forms for injection. Aqueous solutions are generally preferred, based on ease of formulation. However, the compositions can also be suspensions, viscous or semi-viscous gels, or other types of solid or semisolid compositions or sustained release devices or mechanisms that can be injected and/or placed in or around the eye. Aqueous formulations typically can be more than 50%, more than 75%, or more than 90% by weight water.

Additional potential excipients for formulations include solubilizing agents, stabilizing agents, surfactants, demulcents, viscosity agents, diluents, inert carriers, preservatives, binders, and/or disintegrants. Further examples of excipients include certain inert proteins such as albumins; hydrophilic polymers such as polyvinylpyrrolidone; amino acids such as aspartic acid (which may alternatively be referred to as aspartate), glutamic acid (which may alternatively be referred to as glutamate), lysine, arginine, glycine, and histidine; fatty acids and phospholipids such as alkyl sulfonates and caprylate; surfactants such as sodium dodecyl sulphate and polysorbate; nonionic surfactants such as such as TWEEN® (Sigma-Aldrich, St. Louis, Mo.), PLURONICS® (Wyandotte Chemicals Corp., Wyandotte, Mich.), or a polyethylene glycol (PEG) designated 200, 300, 400, or 600; a Carbowax designated 1000, 1500, 4000, 6000, and 10000; carbohydrates such as glucose, sucrose, mannose, maltose, trehalose, and dextrins, including cyclodextrins; polyols such as mannitol and sorbitol; chelating agents such as EDTA; and salt-forming counter-ions such as sodium.

In particular embodiments, in order to prolong the effect of a composition. Compositions can be formulated as sustained-release systems utilizing semipermeable matrices of solid polymers containing at least one administration form. Various sustained-release materials have been established and are well known by those of ordinary skill in the art. Sustained-release systems may, depending on their chemical nature, release active ingredients following administration for a few weeks up to over 100 days.

In particular embodiments, delayed absorption can be accomplished using an oil vehicle. In particular embodiments, administration forms can be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salts. In addition, prolonged absorption of the injectable composition may be brought about by the inclusion of agents which delay absorption such as aluminum monostearate and gelatin.

Injectable depot forms can be made by forming microencapsule matrices of administration forms in biodegradable polymers such as polylactide-polyglycolide. Depending on the ratio of administration form to polymer, and the nature of the particular polymer employed, the rate of administration form release can be controlled. Examples of other biodegradable polymers include poly(orthoesters) and poly(anhydrides). Injectable depot formulations are also prepared by entrapping nucleic acid construct(s) in liposomes or microemulsions which are compatible with body tissue.

Alternatively, delayed absorption of a composition can be accomplished by the use of a liquid suspension of crystalline or amorphous material having poor water solubility. The rate of release then depends upon rate of dissolution which, in turn, may depend upon crystal size and crystalline form.

For administration by inhalation (e.g., nasal or pulmonary), the compositions can be formulated as aerosol sprays for pressurized packs or a nebulizer, with the use of suitable propellants, e.g. dichlorodifluoromethane, trichlorofluoromethane, or dichlorotetra-fluoroethane.

Any composition described herein can advantageously include any other pharmaceutically acceptable carriers which include those that do not produce significantly adverse, allergic, or other untoward reactions that outweigh the benefit of administration, whether for research, prophylactic, and/or therapeutic treatments. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by U.S. FDA Office of Biological Standards and/or other relevant foreign regulatory agencies.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants. Fillers and excipients are commercially available from companies such as Aldrich Chemical Co., FMC Corp, Bayer, BASF, Alexi Fres, Witco, Mallinckrodt, Rhodia, ISP, and others.

The present disclosure further provides for kits including one or more nucleic acid constructs for practicing any of the methods described herein. The kits may include a notice in the form prescribed by a governmental agency regulating the manufacture, use or sale of pharmaceuticals, biological products, lab developed tests, etc., which notice reflects approval by the agency of the manufacture, use or sale for human administration and/or testing. Treatment portions of the kits may include a composition described herein in a ready-to-use form and/or a form that requires preparation before administration (e.g., lyophilized). The kits may also include syringes, pipettes, antiseptics, tubing, gloves, diluents, etc. as well as instructions for practicing any method described herein which may include relevant reference levels.

In particular embodiments, the current disclosure utilizes the nucleic acid construct, compositions and/or kits disclosed herein to treat a subject with a disease, such as DM and DR. The DM can be Type 1 diabetes, Type 2 diabetes or gestational diabetes. The DR can be NPDR or PDR.

Subjects include humans, veterinary animals (dogs, cats, reptiles, birds, etc.) livestock (horses, cattle, goats, pigs, chickens, etc.) and research animals (monkeys, rats, mice, fish, etc.). Subjects in need thereof include subjects diagnosed with a form of DM, DR, or an age-related disease such as Alzheimer's disease or Parkinson's disease.

Treating a subject includes administering a therapeutically effective amount of a composition to the subject. Therapeutically effective amounts include those that provide effective amounts, prophylactic treatments and/or therapeutic treatments.

An “effective amount” is the amount of active agent(s) (e.g., nucleic acid construct(s) and/or vector(s)) or composition(s) necessary to result in a desired physiological change in vivo or in vitro. Effective amounts are often administered for research purposes. As an example, effective amounts disclosed herein reduce cellular oxidative stress, inflammation and/or apoptosis, in particular embodiments, cellular oxidative stress, inflammation and/or apoptosis associated with DM, DR, or an age-related disease. These endpoints can be measured by ELISA to determine the level of oxidative and nitrosative stress markers such as 8-oxo-deoxyGuanosine and Nitrotryrosine as well as H₂O₂ assays. Inflammation can be measured by Quantitive PCR, ELISA or Western Blot by examining inflammatory markers including NLRP3, interleukn-1 beta (IL-1β), tumor necrosis factor alpha (TNF-α), iNOS, or intercellular adhesion molecule 1 (ICAM1). Cell death can be determined by DNA nick assay or TUNEL in tissue or cell culture by Immunohistochemistry (IHC) or other cell culture methods such trypan blue methods.

A prophylactic treatment is administered to a subject that has been diagnosed with DM, DR, or an age-related disease, but does not yet display significant symptoms or complications of the diagnosis. For example, in relation to a DM diagnosis, the subject might show hyperglycemia, but does not yet display complications (e.g., DR) associated with the diagnosis. In relation to age-related disorders, the subject might show anatomical changes or inflammatory changes, with not-yet-existent or mild behavioral effects. Prophylactic treatments are administered to delay the onset of and/or reduce the severity of a condition before it fully emerges.

A therapeutic treatment is administered to a subject that has been diagnosed with DM, DR, or an age-related disease and displays complications associated with the diagnosis.

Therapeutic treatments reduce or reverse, delay or prevent the worsening of symptoms. For example, in relation to a DM diagnosis, therapeutic treatments can reduce or reverse, delay or prevent symptoms such as hyperglycemia and/or reduce the severity of diabetic complications. In relation to age-related disorders, therapeutic treatments can reduce or reverse, delay or prevent symptoms such as memory, mood, and/or motor impairments.

Both prophylactic and therapeutic treatments can provide anti-hyperglycemic effect and/or anti-diabetic effects.

An anti-hyperglycemic effect refers to normalization of a subject's blood glucose level. The normal blood glucose level in an adult is less than 100 mg/dL after fasting for at least 8 hours and 140 mg/dL within two hours after a meal. A blood glucose level higher than 100 mg/dL after fasting for 8 hours or higher than 140 mg/dL within two hours after a meal indicates that the subject is hyperglycemic or diabetic. Blood glucose can be measured using commercially-available kits.

An anti-diabetic effect refers to the alleviation of a symptom or complication of diabetes, such as delayed wound healing, vision loss, inflammation of the retina and/or retinal gliosis. Additional symptoms or complications of diabetes include microvascular complications of the kidney (diabetic nephropathy, DN). Anti-diabetic effects also include reduced cellular oxidative stress, inflammation and/or apoptosis associated with DM or DR.

A vitreous sample may be used to measure oxidative and inflammatory markers related to ocular complications. A delay in the development of microaneurysms in the retina of diabetics under ophthalmoscopic examination can reveal the effectiveness of treatment. Furthermore, other retinal neurological functions such as retinal electroradiogram (ERG) and Optical Coherence Tomography (OCT) can be used to investigate retinal neurovascular degeneration in combination with retinal angiophrapy for diabetic retinopathy, which generally may occur earlier than actual blood vessel damage. Early DN can be measured by urine albuminurea, leakage of albumin and/or protein in the kidney and their presence in the urine.

The compositions described herein can be administered parenterally, such as intramuscularly, subcutaneously, intramedullary, intrathecally, direct intraventricularly, intravenously, intraperitoneally, intranasally, intraocularly, intravitreally, retinally, or subretinally. As an example, for the treatment of DR, a composition including a nucleic acid encoding an iRNA targeting VEGF-A under the control of a TXNIP promoter can be delivered (e.g., injected) at the site of neovascularization in the vitreous of the eye. Alternatively or additionally, genetically-modified cells expressing insulin or Trx under the control of a TXNIP promoter can be administered (e.g., injected) subcutaneously.

Alzheimer's disease (AD) is a neurodegenerative disorder of the central nervous system and the leading cause of a progressive dementia in the elderly population. Its clinical symptoms are impairment of memory, cognition, temporal and local orientation, judgment and reasoning but also severe emotional disturbances. AD is characterized by 2 major pathologies in the central nervous system (CNS), the occurrence of amyloid plaques and neurofibrillar tangles. Neurofibrillar tangles are intracellular aggregates of the micro tubule-associated protein tau (MAPT). Amyloid plaques occur in the extracellular space; their principal components are Aβ-peptides.

Parkinson's disease (PD) is a degenerative and inflammatory disorder of the central nervous system. Four motor symptoms are considered hallmarks of PD: tremor, rigidity, slowness of movement, and postural instability. Later in disease progression, thinking and behavioral problems may arise and can range from mild to severe, with dementia commonly occurring in the advanced stages of the disease. Depression is the most common psychiatric symptom. Other common symptoms include disorders of speech, cognition, mood, behavior, and thought. Cognitive disturbances further include executive dysfunction, which can include problems with planning, cognitive flexibility, abstract thinking, rule acquisition, initiating appropriate actions and inhibiting inappropriate actions, selecting relevant sensory information, fluctuations in attention, slowed cognitive speed, and memory loss. Other symptoms include sleep disturbances.

In particular embodiments, age-related conditions with a central nervous system component can be evaluated using tests for cognitive impairment, and/or neuropsychiatric morbidities, such as disorders of cognitive function, memory, mood, behavior, thought, REM Sleep Behavior Disorder, apathy, fatigue, indifference and lack of social engagement, and dullness. Methods of measuring and monitoring these aspects are known in the art and include, for example, serial position testing which focuses on human memory processes (Surprenant, Perception and Psychophysics, 63(4): 737-745 (2001)), word superiority testing which focuses on human speech and language (Krueger, Memory & Cognition, 20(6):685-694 (1992)), the Brown-Peterson test which focuses on human short-term memory (Nairne, et al., Quarterly Journal of Experimental Psychology A: Human Experimental Psychology, 52:241-251 (1999)), memory span testing (May, et al., Memory & Cognition, 27(5):759-767 (1999)), visual search testing (Wolfe, et al., Journal of Experimental Psychology: Human Perception and Performance, 15(3):419-433 (1989)), and knowledge representation (e.g., semantic network) testing. Additional tests examine processing speed, reaction time, i.e. clock speed; flexibility and ability to adapt to changes in task rules; attention, focus and concentration; problem solving; memory; and verbal fluency. Representative tests and instruments include traditional IQ tests like the WAIS and Progressive Ravens Matrices, and the battery of tests available through Luminosity (Lumos Labs, Inc.).

As indicated, the methods disclosed herein for treating a disease (e.g., DM, DR, or an age-related disease) can include genetic therapies including ex vivo and in vivo genetic therapies. Genetic therapies can be achieved using any method known in the art and described above, including the use of viral vectors and nonviral vectors (particularly for in vivo genetic therapies).

In ex vivo genetic therapy, a nucleic acid construct described herein is introduced into cells to produce and secrete the protein or iRNA encoded by the gene under the control of a TXNIP promoter. Subsequently, these cells producing the encoded proteins or iRNA can be transplanted into a subject in need of treatment. In particular embodiments, the cells are harvested from the subject prior to introducing the nucleic acid construct described herein for performing transplantation. Following genetic modification, the subject's cells are re-introduced to the subject, for example, subcutaneously.

The term “transplantation” refers to any method of transferring a cell to a subject. Transplantation can involve direct injection of a suspension of cells into a relevant site such as the subcutaneous layer or the bloodstream of a subject. Surgical implantation of a cell mass into a tissue or organ of a subject, or perfusion of a tissue or organ of the subject with a cell suspension can also be performed.

The procedure for transplantation will be determined by the need of the cell to reside in a particular tissue or organ and by the ability of the cell to find and be retained by the target tissue or organ. Optimization of transplantation conditions and procedures can have substantial effects on the cell fate of implanted cells. Transplantation or cell implantation techniques may be adapted to particular subjects.

In particular embodiments, a nucleic acid encoding (i) insulin, IGF-1 gene, PDX1, or a neurotrophic factor such as brain-derived neurotrophic factor (BDNF) or glia-derived neurotrophic factor (GDNF); and/or Trx and/or (ii) an iRNA reducing expression of TXNIP, VEGF, iNOS, HIF-1alpha, and/or NLRP3 under the control of a TXNIP promoter is introduced into cells (e.g, non-beta cells such as mesenchymal cells, pre-adipocytes, adipocytes, fibroblasts, or muscle cells) for treatment of a disease (e.g., DM, DR, or an age-related disease). The cells can be xenogenic cells, allogenic cells, isogenic cells, or autologous cells in relation to the subject in need of treatment. In autologous transplantation, the cells (e.g., adipose cells) can be harvested from the subject and expanded in culture before or following the genetic modification.

For administration, therapeutically effective amounts (also referred to herein as doses) can be initially estimated based on results from in vitro assays and/or animal model studies. Such information can be used to more accurately determine useful doses in subjects of interest.

The actual dose amount administered to a particular subject can be determined by a physician, veterinarian, or researcher taking into account parameters such as physical, physiological and psychological factors including target, body weight, stage of DM, DR, or an age-related disease, the type of DM (type 1, type 2, type 3, or gestational) or DR (NPDR or PDR), type or stage of age-related condition, previous or concurrent therapeutic interventions, idiopathy of the subject, and route of administration.

Exemplary doses can include 0.0001 mg/kg to 100 mg/kg of a nucleic acid construct disclosed herein. The total daily dose can be 0.1 mg/kg to 50.0 mg/kg of the nucleic acid construct administered to a subject one to three times a day. Additional useful doses can often range from 0.1 to 5 μg/kg or from 0.5 to 1 μg/kg. In other examples, a dose can include 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1000 μg/kg, 0.1 to 5 mg/kg or from 0.5 to 1 mg/kg. In other examples, a dose can include 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg.

Exemplary cell doses for genetic therapies can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

In particular embodiments, doses can be administered repeatedly over a range of time periods. It can be administered daily, once every few days, weekly, or monthly. The timing of administration can vary from subject to subject, depending upon such factors as the severity of a subject's symptoms and the stage and type of diabetes or age-related condition. For example, therapeutically effective amounts can be achieved by administering single or multiple doses during the course of a treatment regimen (e.g., every other day, every 3 days, every 4 days, every 5 days, every 6 days, weekly, every 2 weeks, every 3 weeks, or monthly). In particular embodiments, doses can be administered to a subject once a month for an indefinite period of time, or until the subject no longer requires therapy. In addition, sustained release compositions containing the doses can be used to maintain a relatively constant dosage in the site of delivery.

In particular embodiments, treatments disclosed herein can be administered in combination with a secondary medication. For example, the secondary medication can be a supplemental treatment for DM or DR (e.g., insulin or metformin). The secondary medication could also be an anesthetic, such as ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and/or phenazopyridine.

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Exemplary Embodiments

0. A nucleic acid construct including a thioredoxin-interacting protein (TXNIP) promoter operably linked to a gene encoding a therapeutic protein or an interfering RNA (iRNA) sequence.
1. A nucleic acid construct including a thioredoxin-interacting protein (TXNIP) promoter operably linked to a gene encoding (i) a therapeutic protein selected from (a) insulin, an insulin-like protein, or an insulin-promoting protein; (b) a neurotrophic factor selected from brain-derived neurotrophic factor (BDNF) or glia-derived neurotrophic factor (GDNF); or (c) a protein that reduces cellular oxidative stress, inflammation and/or apoptosis; and/or (ii) an interfering nucleic acide (e.g., iRNA) that targets expression of a protein that promotes cellular oxidative stress, inflammation and/or apoptosis.
2. A nucleic acid construct of embodiment 0 or 1 wherein the therapeutic protein includes insulin, IGF-1, PDX1, BDNF, GDNF or Trx.
3. A nucleic acid construct of any of embodiments 0-2 wherein the promoter includes SEQ ID NO: 1, SEQ ID NO: 28, SEQ ID NO: 29 and/or SEQ ID NO: 30.
4. A nucleic acid construct of any of embodiments 0-3 wherein the gene includes SEQ ID NO:

2.

5. A nucleic acid construct of any of embodiments 0-3 wherein the construct includes SEQ ID NO: 3.
6. A nucleic acid construct of any of embodiments 0-5 wherein the gene encodes SEQ ID NO: 4, SEQ ID NO: 5, SEQ ID NO: 6, SEQ ID NO: 7, SEQ ID NO: 8, SEQ ID NO: 44 or SEQ ID NO: 46.
7. A nucleic acid construct of any of embodiments 0-6 wherein an iRNA sequence targets expression of TXNIP, VEGF, iNOS, HIF-1alpha, and/or NLRP3.
8. A nucleic acid construct of any of embodiments 0-6 wherein an iRNA sequence targets expression of SEQ ID NO: 9, SEQ ID NO: 10, SEQ ID NO: 11, SEQ ID NO: 12, and/or SEQ ID NO: 13.
9. A nucleic acid construct of any of embodiments 0-8 wherein an iRNA sequence includes SEQ ID NO: 14, SEQ ID NO: 15, SEQ ID NO: 16, SEQ ID NO: 17, SEQ ID NO: 18, SEQ ID NO: 19, and/or SEQ ID NO: 20.
10. A nucleic acid of any of embodiments 0-9 linked to a cell penetrating peptide.
11. A nucleic acid of embodiment 10 wherein the cell penetrating peptide is selected from a transportan peptide, a TP10 peptide, a pVEC peptide, a penetratin peptide, a tat fragment peptide, a signal sequence based peptide, or an amphiphilic model peptide.
12. A nucleic acid of embodiment 10 wherein the cell penetrating peptide is selected from SEQ ID NO: 21, SEQ ID NO: 22, SEQ ID NO: 23, SEQ ID NO: 24, SEQ ID NO: 25, SEQ ID NO: 26, or SEQ ID NO: 27.
13. A vector including a nucleic acid construct of any one of embodiments 0-9.
14. A vector of embodiment 13, wherein the vector further includes regulatory elements.
15. A cell including a nucleic acid construct of any of embodiments 0-12 and/or a vector of embodiments 13 or 14.
16. A cell of embodiment 15, wherein the cell is selected from a stem cell, a mesenchymal cell, a pre-adipocyte, an adipocyte, a hepatocyte, a fibroblast, or a muscle cell.
17. A composition including a nucleic acid construct of any one of embodiments 0-12 a vector of embodiments 13 or 14 or a cell of embodiments 15 or 16 and a pharmaceutically acceptable carrier.
18. A composition of embodiment 17 formulated for injection.
19. A composition of embodiment 17 formulated for subcutaneous, sub-scleral, or intravitreal injection.
20. A composition of embodiment 17 or 18 formulated for intraocular administration.
21. A method of treating DM, DR, or an age-related condition in a subject in need thereof including administering a therapeutically effective amount of a nucleic acid construct, vector, cell or composition of any of embodiments 0-20 to the subject, thereby treating DM, DR, or the age-related condition in the subject.
22. A method of embodiment 21 wherein the administering treats DM and/or DR.
23. A method of embodiment 21 or 22 wherein the administering treats Alzheimer's disease and/or Parkinson's disease.
24. A method of any of embodiments 21-23 wherein the treating provides a prophylactic treatment or a therapeutic treatment.
25. A method of embodiment 24 wherein the prophylactic treatment or the therapeutic treatment is evidenced by one or more of an anti-hyperglycemic effect and/or an anti-diabetic effect.
26. A method of any of embodiments 21-25 wherein the administering is sub-scleral or intravitreal injection.
27. A method of any of embodiments 21-25 wherein the administering is subcutaneous injection.
28. A method of any of embodiments 21-27 wherein the administered nucleic acid is within a cell obtained from the subject.
29. A method of embodiment 28 wherein the cell obtained from the subject is an adipocyte.
30. A method of up-regulating insulin production in a high glucose environment including introducing a nucleic acid construct, vector or composition of any one of embodiments 0-14 or 17-20 into a cell wherein following the introduction the cell produces an increased amount of insulin in the high glucose environment as compared to a control cell without the introduction in the high glucose environment.
31. A method of embodiment 30 wherein the high glucose environment is within a diabetic subject.
32. A method of embodiment 30 wherein the high glucose environment is a blood glucose level higher than 100 mg/dL after fasting for 8 hours or higher than 140 mg/dL within two hours after a meal.
33. A method of embodiment 30 wherein the cells are selected from a stem cell, a mesenchymal cell, a pre-adipocyte, an adipocyte, a hepatocyte, a fibroblast, or a muscle cell.
34. A method of any of embodiments 21-33 wherein the methods includes genetically-modifying a cell with a nucleic acid construct or vector of any embodiments 1-13. Examples. Background. TXNIP is highly induced by diabetes and high glucose in most cell types examined so far. Its induction occurs within minutes (15-30 minutes) and is suppressed by insulin or IGF-
35. The TXNIP promoter is in an open or poised configuration that high glucose and its metabolites including hexosamines can induce TXNIP transcription and translation quickly. The fact that the TXNIP promoter and gene expression is highly induced by high glucose and causes insulin resistance, may involve inhibition of insulin signaling by targeting Akt and/or PTEN. A lack of or defect in insulin signaling may be critical in TXNIP overexpression and immature cell death in diabetes. Therefore, by employing the TXNIP promoter, one can introduce a gene or non-coding RNA (microRNA, shRNA, or long non-coding RNA) to alter disease-associated gene expressions, such as in DM and DR.

The promoter of TXNIP can be used to deliver genes and non-coding RNAs for gene therapy in diabetes and its complications. TXNIP binds to Trx and inhibits is thiol reducing and anti-oxidant function causing cellular oxidative stress and apoptosis. In addition, TXNIP is also considered as a homologue of a-arrestin, which is involved in protein scaffolding, receptor endocytosis and trafficking independently of Trx binding. TXNIP-promoter linked gene and non-coding RNA plasmids are developed to deliver gene expression or knock down.

As an example, a TXNIP promoter and insulin gene construct was developed and expressed in beta-cell or non-beta cells for insulin gene therapy in diabetics and a TXNIP-promoter linked with TXNIP shRNA was developed to blunt TXNIP expression itself (Fire Fights Fire, F3) in diabetes and its complications. As another example, a construct including TXNIP promoter linked to VEGF-A shRNA is developed for gene therapy via VEGF-A targeting in proliferative diabetic retinopathy, age-related macular dystrophy, and retinopathy of prematurity and to blunt cancer angiogenesis. Using this TXNIP promoter gene/shRNA technology and tissue engineering approaches, genes can be targeted to treat diseases such as DM and DR.

### Example 1: Cloning of Nucleic Acid Construct Including TXNIP Promoter Operably Linked to Thioredoxin 1

A rat TXNIP promoter Trx1 gene construct (FIG. 2) was cloned into a pcDNA3.1/Hygro(+) plasmid. Particularly, the CMV promoter from pcDNA.3.1 was cut out with NruI-ApaI and SEQ ID NO: 31 was inserted. This plasmid was custom-prepared by Gene Script, Piscataway, N.J.

### Example 2. The TXNIP Promoter is Activated by High Glucose

As shown in FIGS. 15A-15C, 16A, and 16B, the expression of Trx1 in rMC1 cells driven by the TXNIP promoter under high glucose conditions. It is proposed that this neutralizes TXNIP activity, thereby reducing cellular oxidative stress and apoptosis.

Transfection and selection of rMC1 expressing the TXNIP.promoter Trx-1 gene. rMC1 cells were grown upto 90-95% confluence in a DMEM/H-12 medium in a 6-well culture plate containing 5% serum at 37° C. in a humidifier. OptiPro SFM and Lipfectamine 2000 CD were used for transfecting 1 ug/ul cDNA in a final 250 ul according instructions from Invitrogen (Cat #12566-014). Antibiotics were omitted from the media. Transfection was continued for 6 h in a humidified CO2 incubation. The media were changed to full growth medium containing 5% serum and antibiotics. After 24 h, the transfected cells were trypsinized and sub-cultured to confluency (48 h) and they were subsequently selected by hygromycin B using a 200 ug/ml concentration.

Intracellular reactive oxygen species (ROS) measurement. The formation of intracellular ROS in cells can be detected by using the fluorescent probe, 5-(and-6)-chloromethyl-2′,7′-dichlorodihydrofluorescein diacetate, acetyl ester (CM-H2DCFDA). This dye can enter living cells by passive diffusion and it is non-fluorescent until the acetate group is cleaved off by intracellular esterase and oxidation occurs within the cell. Approximately 1×10⁵ cells/ml were cultured in 24 well plates, serum-starved overnight and glucose were added for the specified time period. Then, CM-H2DCFDA (10 μM) was incubated for 60 min at 37° C. The medium with the dye was aspirated (to remove the extracellular dye), washed with PBS (3×), then the PBS is added to cells. The fluorescence was measured in a Gemini Fluorescent Microplate Reader (Molecular Devices) with the bottom read scanning mode at 480 nm excitation and emission at 530 nm. We propose that TXNIP.promoter Trx-1 cells will have less oxidative stress under high glucose exposure.

DNA fragmentation detection in apoptotic cells. To measure DNA damage under high glucose in rMC1 TXNIP.promoter Trx-1 overexpressing cells, (i) IHC to detect chromatin fragmentation and condensation using DAPI staining of the nucleus as described above in IHC section and (ii) single cell gel electrophoresis (SCGE) or the alkaline comet assay using the OxiSelect™ Comet Assay kit (cat# STA-350) from Cell Biolabs, Inc (San Diego, Calif.) were used. The SCGE or Comet assay is a useful method to measure DNA damage in individual cells under an electrophoretic field. For this, rMC1 cells were cultured in 6 well plates and maintained in LG or HG conditions for 3 or 5 days. Cells were scrapped off and resuspended in cold-PBS (without Mg2+ and Ca2+) at 1×105 cells/ml. Cell suspension (10 μl) and 90 μl of Comet Agarose (melted at 90° C. and maintained at 37° C.) were mixed and immediately pipetted (75 μl) on the OxiSelect™ Comet Slide. The slides were kept at 4° C. for 15 min in the dark. The slides were then carefully immersed (while maintaining horizontal position to prevent agarose slipping off the slide) in the pre-chilled lysis buffer in a small container (25 ml) for 30 min at 4° C. in the dark. The slides were then transferred to an electrophoresis chamber and filled with cold Alkaline Electrophoresis Solution (300 mM NaOH, 1 mM EDTA, pH 13). Electrophoresis was run at 20 V (1 V/cm for 20 cm apart chamber electrodes) for 25 min. After completing the electrophoresis, the agarose slide was carefully transferred to a container and immerged in pre-chilled sterile H₂O for 2 min, aspirated and repeated two times. Then the slides were emerged again in 70% alcohol for 5 min and air dried for 30 min. After the agarose is completely dried, 100 μl of a diluted Vista Green DNA Dye was added and kept for 15 min at room temperature. The slide is then view under OLYMPUS BX 51 fluorescence microscope for green fluorescence. The DNA fragmentation is determined by the presence of a Head and a Comet Tail ((displacement of nuclear DNA (head) to a resulting DNA streaking (tail) due to breakage)). It is proposed that TXNIP.promoter Trx-1 expressing cells will have less apoptotic cells under high glucose exposure.

### Example 3: Cloning of Nucleic Acid Construct Including TXNIP Promoter Operably Linked to Thioredoxin 2

As will be understood by one of ordinary skill in the art, each embodiment disclosed herein can comprise, consist essentially of, or consist of its particular stated element, step, ingredient or component. Thus, the terms “include” or “including” should be interpreted to recite: “comprise, consist of, or consist essentially of.” As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would cause a statistically significant reduction in an embodiment's ability to stimulate expression of a therapeutic gene or iRNA disclosed herein in a high glucose environment.

Unless otherwise indicated, all numbers used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to publications, patents and/or patent applications (collectively “references”) throughout this specification. Each of the cited references is individually incorporated herein by reference for their particular cited teachings.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A non-beta cell comprising a nucleic acid construct comprising:
(i) a thioredoxin-interacting protein (TXNIP) promoter operably linked to a gene encoding a therapeutic protein selected from:
(a) insulin, an insulin-like protein, or an insulin-promoting protein,
(b) a neurotrophic factor selected from brain-derived neurotrophic factor (BDNF) or glia-derived neurotrophic factor (GDNF); or
(c) a therapeutic protein that reduces cellular oxidative stress, inflammation and/or apoptosis; or
(ii) a TXNIP promoter operably linked to a gene encoding an interfering nucleic acid sequence that targets expression of a protein that promotes cellular oxidative stress, inflammation and/or apoptosis; or
(iii) both (i) and (ii).

2. The non-beta cell of claim 3, which is a stem cell, a mesenchymal cell, a pre-adipocyte, an adipocyte, a hepatocyte a fibroblast, or a muscle cell.

3. The non-beta cell of claim 1, wherein nucleic acid construct comprises a gene encoding the therapeutic protein insulin, IGF-1, PDX1, or Trx.

4. The non-beta cell of claim 1, wherein nucleic acid construct comprises a gene encoding an interfering nucleic acid sequence that targets expression of TXNIP, VEGF, iNOS, HIF-1alpha, or NLRP3.

5. The non-beta cell of claim 1, wherein the nucleic acid construct further encodes a cell penetrating peptide.

6. The non-beta cell of claim 5, wherein the cell penetrating peptide comprises a transportan peptide, a TP10 peptide, a pVEC peptide, a penetratin peptide, a tat fragment peptide, a signal sequence based peptide, or an amphiphilic model peptide.

7. A composition comprising:
a nucleic acid construct comprising:
(i) a thioredoxin-interacting protein (TXNIP) promoter operably linked to a gene encoding a therapeutic protein selected from:
(a) insulin, an insulin-like protein, or an insulin-promoting protein,
(b) a neurotrophic factor selected from brain-derived neurotrophic factor (BDNF) or glia-derived neurotrophic factor (GDNF); or
(c) a therapeutic protein that reduces cellular oxidative stress, inflammation and/or apoptosis; or
(ii) a TXNIP promoter operably linked to a gene encoding an interfering nucleic acid sequence that targets expression of a protein that promotes cellular oxidative stress, inflammation and/or apoptosis; and
a pharmaceutically acceptable carrier.

8. The composition of claim 7 formulated for injection.

9. The composition of claim 7 formulated for subcutaneous, sub-scleral, or intravitreal injection.

10. The composition of claim 7 formulated for intraocular administration.

11. The composition of claim 17, wherein nucleic acid construct comprises a gene encoding the therapeutic protein insulin, IGF-1, PDX1, or Trx.

12. The composition of claim 17, wherein nucleic acid construct comprises a gene encoding an interfering nucleic acid sequence that targets expression of TXNIP, VEGF, iNOS, HIF-1alpha, or NLRP3.

13. The composition of claim 17, wherein the nucleic acid construct further encodes a cell penetrating peptide.

14. The composition of claim 13, wherein the cell penetrating peptide comprises a transportan peptide, a TP10 peptide, a pVEC peptide, a penetratin peptide, a tat fragment peptide, a signal sequence based peptide, or an amphiphilic model peptide.

15. A method of treating diabetes mellitus (DM) or diabetic retinopathy (DR) in a subject in need thereof comprising: administering to the subject a therapeutically effective amount of a nucleic acid construct comprising:
(i) a thioredoxin-interacting protein (TXNIP) promoter operably linked to a gene encoding a therapeutic protein selected from:
(a) insulin, an insulin-like protein, or an insulin-promoting protein,
(b) a neurotrophic factor selected from brain-derived neurotrophic factor (BDNF) or glia-derived neurotrophic factor (GDNF); or
(c) a therapeutic protein that reduces cellular oxidative stress, inflammation and/or apoptosis; or
(ii) a TXNIP promoter operably linked to a gene encoding an interfering nucleic acid sequence that targets expression of a protein that promotes cellular oxidative stress, inflammation and/or apoptosis; or
(iii) both (i) and (ii),
thereby treating DM or DR in the subject.

16. The method of claim 15, wherein the administering treats DM.

17. The method of claim 15, wherein the administering treats DR.

18. The method of claim 15, wherein the treating provides a prophylactic treatment or a therapeutic treatment.

19. The method of claim 18, wherein the prophylactic treatment or the therapeutic treatment is evidenced by an anti-hyperglycemic effect and/or an anti-diabetic effect.

20. The method of claim 15, wherein the administering comprises sub-scleral or intravitreal injection.

21. The method of claim 15, wherein the administering comprises subcutaneous injection.

22. The method of claim 15, wherein the administered nucleic acid is within a cell obtained from the subject.

23. The method of claim 22, wherein the cell obtained from the subject is an adipocyte.

24. The non-beta cell of claim 15, wherein nucleic acid construct comprises a gene encoding the therapeutic protein insulin, IGF-1, PDX1, or Trx.

25. The non-beta cell of claim 15, wherein nucleic acid construct comprises a gene encoding an interfering nucleic acid sequence that targets expression of TXNIP, VEGF, iNOS, HIF-1alpha, or NLRP3.

26. The non-beta cell of claim 15, wherein the nucleic acid construct further encodes a cell penetrating peptide.

27. The non-beta cell of claim 26, wherein the cell penetrating peptide comprises a transportan peptide, a TP10 peptide, a pVEC peptide, a penetratin peptide, a tat fragment peptide, a signal sequence based peptide, or an amphiphilic model peptide.

28. A method of up-regulating insulin production in a high glucose environment comprising:
introducing into a cell a nucleic acid construct comprising:
(i) a thioredoxin-interacting protein (TXNIP) promoter operably linked to a gene encoding a therapeutic protein selected from:
(a) insulin, an insulin-like protein, or an insulin-promoting protein,
(b) a neurotrophic factor selected from brain-derived neurotrophic factor (BDNF) or glia-derived neurotrophic factor (GDNF); or
(c) a therapeutic protein that reduces cellular oxidative stress, inflammation and/or apoptosis; or
(ii) a TXNIP promoter operably linked to a gene encoding an interfering nucleic acid sequence that targets expression of a protein that promotes cellular oxidative stress, inflammation and/or apoptosis; or
(iii) both (i) and (ii),
wherein following the introduction the cell produces an increased amount of insulin in the high glucose environment as compared to a control cell, into which the nucleic acid construct has not been introduced, in the high glucose environment.

29. The method of claim 28, wherein the high glucose environment is within a diabetic subject.

30. The method of claim 28, wherein the high glucose environment is a blood glucose level higher than 100 mg/dL after fasting for 8 hours or higher than 140 mg/dL within two hours after a meal.

31. The method of claim 28, wherein the cells are selected from a stem cell, a mesenchymal cell, a pre-adipocyte, an adipocyte, a hepatocyte, a fibroblast, or a muscle cell.

32. The non-beta cell of claim 28, wherein nucleic acid construct comprises a gene encoding the therapeutic protein insulin, IGF-1, PDX1, or Trx.

33. The non-beta cell of claim 28, wherein nucleic acid construct comprises a gene encoding an interfering nucleic acid sequence that targets expression of TXNIP, VEGF, iNOS, HIF-1alpha, or NLRP3.

34. The non-beta cell of claim 28, wherein the nucleic acid construct further encodes a cell penetrating peptide.

35. The non-beta cell of claim 34, wherein the cell penetrating peptide comprises a transportan peptide, a TP10 peptide, a pVEC peptide, a penetratin peptide, a tat fragment peptide, a signal sequence based peptide, or an amphiphilic model peptide.


SEQUENCE LISTING (47 sequences)

SEQ ID NO: 1 - DNA, 1596 bp, Rattus norvegicus
acgcctaataattctgcccaaatatggaaggaggctaggactcaatgacaaggctctggcggggtggaggtgggaggaggtgggtgaggaatagggtaggggccctggggtgtcagggagtggtgggtagtccttcaacatacaagagtttctttgcactcaagttatttctctagtcagctcctgaagcatttctcagcaaggtttgccaaatagccaagtgaaaccaatacagcttcagccctggggagactgaaacaggctgaggggtatgcatttcatttagtgattttgatgagaggacaaatggggaaaaaaaaaaagagtgacaggaactcgggaacaaagtaaggagtgacagatcgttttcttcctttttcttcccccctgtttgtttgtttgtacccaccccttgtttcctggaggagcagggacgaagggagagatcagtgtaaaggtacacacctcactaaagctacagtgaggtaaataagggaacatatacaaaatgttccccaacctcacaggtacactgaagagatgaggggataagcaacaggatgtggacactcccttactgcttccgttccagagaacagaacagaatagaacgtaatgggcgaggaacaatagcagcacatagggcatggaacgaaggggtacacaccagaccatgtaccaataaggactttaagccagacttaaaatatctgacaagtcccccgcccccgggagatggaagcgttttattcaatagaagtgtaatgggagcacatcgggtaggctctttctcactaacacaactgcactctcgccctccgctccatcctgaagtatccttggggattgtttttcacagacttgcgaacttgtgagccaggaataaatggtcacgtcgaaatgaattgcgctggctaagacaggcatgaaatcctctcctaagcacatttttcttttacctaaaaaaagaaggggaaaaaaaccaacaaagcacacacccaaacaacccagctcccgagaggagtaccctggatgaggttcagggtctcggggtcccagactcccgagggagccacccgctgccccaggccccgcccctcctccctggcaaggctgcgcacccgaacaacaaccattttccccgctaagagcacactgtgtccacgcgcctctgcggcctcgctgattggttagaggcctggtaaacaaggaccaagtagccaatgggagaactgtgcacgagggatgcacgagcctccgggccagcactcgcgtggagcgtcaagccaggcggctatataatgccgtttccggctcccgcttgacactctcctcttctggtctttggatatccagagttcctccagttgcgagaaaacagctgttatttttctctgaaagcttttggcacaaccagctggttgaaactttcaggcaccttttagagaagttgttaaggttttgtttgaggctttctttgggtttttaagccctctctgcctcacggagagacttaagttcttagtttgctgagaaggattctgaagagtttttcctctccggcttccgtttttcttgaacccactcggctcaatc

SEQ ID NO: 2 - DNA, 330 bp, Rattus norvegicus
atggtgaagctgatcgagagcaaggaagcttttcaggaggccctggccgctgcgggagacaagcttgtggtagtggacttctctgccacgtggtgtggaccttgcaaaatgatcaagcccttctttcattccctctgtgacaagtattccaatgtggtgttccttgaagtagacgtggatgactgccaggatgttgctgcagactgtgaagtcaaatgcatgccgaccttccagttctataaaaagggtcaaaaggttggggagttctctggtgctaacaaggaaaagctcgaagccactattacggagtttgcctaatcatgctctgaa

SEQ ID NO: 3 - DNA, 1938 bp, Artificial Sequence
  TXNIP promoter Trx1 cDNA construct
tcgcgaacgcctaataattctgcccaaatatggaaggaggctaggactcaatgacaaggctctggcggggtggaggtgggaggaggtgggtgaggaatagggtaggggccctggggtgtcagggagtggtgggtagtccttcaacatacaagagtttctttgcactcaagttatttctctagtcagctcctgaagcatttctcagcaaggtttgccaaatagccaagtgaaaccaatacagcttcagccctggggagactgaaacaggctgaggggtatgcatttcatttagtgattttgatgagaggacaaatggggaaaaaaaaaaagagtgacaggaactcgggaacaaagtaaggagtgacagatcgttttcttcctttttcttcccccctgtttgtttgtttgtacccaccccttgtttcctggaggagcagggacgaagggagagatcagtgtaaaggtacacacctcactaaagctacagtgaggtaaataagggaacatatacaaaatgttccccaacctcacaggtacactgaagagatgaggggataagcaacaggatgtggacactcccttactgcttccgttccagagaacagaacagaatagaacgtaatgggcgaggaacaatagcagcacatagggcatggaacgaaggggtacacaccagaccatgtaccaataaggactttaagccagacttaaaatatctgacaagtcccccgcccccgggagatggaagcgttttattcaatagaagtgtaatgggagcacatcgggtaggctctttctcactaacacaactgcactctcgccctccgctccatcctgaagtatccttggggattgtttttcacagacttgcgaacttgtgagccaggaataaatggtcacgtcgaaatgaattgcgctggctaagacaggcatgaaatcctctcctaagcacatttttcttttacctaaaaaaagaaggggaaaaaaaccaacaaagcacacacccaaacaacccagctcccgagaggagtaccctggatgaggttcagggtctcggggtcccagactcccgagggagccacccgctgccccaggccccgcccctcctccctggcaaggctgcgcacccgaacaacaaccattttccccgctaagagcacactgtgtccacgcgcctctgcggcctcgctgattggttagaggcctggtaaacaaggaccaagtagccaatgggagaactgtgcacgagggatgcacgagcctccgggccagcactcgcgtggagcgtcaagccaggcggctatataatgccgtttccggctcccgcttgacactctcctcttctggtctttggatatccagagttcctccagttgcgagaaaacagctgttatttttctctgaaagcttttggcacaaccagctggttgaaactttcaggcaccttttagagaagttgttaaggttttgtttgaggctttctttgggtttttaagccctctctgcctcacggagagacttaagttcttagtttgctgagaaggattctgaagagtttttcctctccggcttccgtttttcttgaacccactcggctcaatcatggtgaagctgatcgagagcaaggaagcttttcaggaggccctggccgctgcgggagacaagcttgtggtagtggacttctctgccacgtggtgtggaccttgcaaaatgatcaagcccttctttcattccctctgtgacaagtattccaatgtggtgttccttgaagtagacgtggatgactgccaggatgttgctgcagactgtgaagtcaaatgcatgccgaccttccagttctataaaaagggtcaaaaggttggggagttctctggtgctaacaaggaaaagctcgaagccactattacggagtttgcctaatcatgctctgaagggccc

SEQ ID NO: 4 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 5 - protein, 158 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGSTFEERK

SEQ ID NO: 6 - protein, 283 aa, Homo sapiens
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 7 - protein, 105 aa, Homo sapiens
MVKQIESKTAFQEALDAAGDKLVVVDFSATWCGPCKMINPFFHSLSEKYSNVIFLEVDVDDCQDVASECEVKCTPTFQFFKKGQKVGEFSGANKEKLEATINELV

SEQ ID NO: 8 - protein, 166 aa, Homo sapiens
MAQRLLLRRFLASVISRKPSQGQWPPLTSKALQTPQCSPGGLTVTPNPARTIYTTRISLTTFNIQDGPDFQDRVVNSETPVVVDFHAQWCGPCKILGPRLEKMVAKQHGKVVMAKVDIDDHTDLAIEYEVSAVPTVLAMKNGDVVDKFVGIKDEDQLEAFLKKLIG

SEQ ID NO: 9 - protein, 391 aa, Homo sapiens
MVMFKKIKSFEVVFNDPEKVYGSGEKVAGRVIVEVCEVTRVKAVRILACGVAKVLWMQGSQQCKQTSEYLRYEDTLLLEDQPTGENEMVIMRPGNKYEYKFGFELPQGPLGTSFKGKYGCVDYWVKAFLDRPSQPTQETKKNFEVVDLVDVNTPDLMAPVSAKKEKKVSCMFIPDGRVSVSARIDRKGFCEGDEISIHADFENTCSRIVVPKAAIVARHTYLANGQTKVLTQKLSSVRGNHIISGTCASWRGKSLRVQKIRPSILGCNILRVEYSLLIYVSVPGSKKVILDLPLVIGSRSGLSSRTSSMASRTSSEMSWVDLNIPDTPEAPPCYMDVIPEDHRLESPTTPLLDDMDGSQDSPIFMYAPEFKFMPPPTYTEVDPCILNNNVQ

SEQ ID NO: 10 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 11 - protein, 1153 aa, Homo sapiens
MACPWKFLFKTKFHQYAMNGEKDINNNVEKAPCATSSPVTQDDLQYHNLSKQQNESPQPLVETGKKSPESLVKLDATPLSSPRHVRIKNWGSGMTFQDTLHHKAKGILTCRSKSCLGSIMTPKSLTRGPRDKPTPPDELLPQAIEFVNQYYGSFKEAKIEEHLARVEAVTKEIETTGTYQLTGDELIFATKQAWRNAPRCIGRIQWSNLQVFDARSCSTAREMFEHICRHVRYSTNNGNIRSAITVFPQRSDGKHDFRVWNAQLIRYAGYQMPDGSIRGDPANVEFTQLCIDLGWKPKYGRFDVVPLVLQANGRDPELFEIPPDLVLEVAMEHPKYEWFRELELKWYALPAVANMLLEVGGLEFPGCPFNGWYMGTEIGVRDFCDVQRYNILEEVGRRMGLETHKLASLWKDQAVVEINIAVLHSFQKQNVTIMDHHSAAESFMKYMQNEYRSRGGCPADWIWLVPPMSGSITPVFHQEMLNYVLSPFYYYQVEAWKTHVWQDEKRRPKRREIPLKVLVKAVLFACMLMRKTMASRVRVTILFATETGKSEALAWDLGALFSCAFNPKVVCMDKYRLSCLEEERLLLVVTSTFGNGDCPGNGEKLKKSLFMLKELNNKFRYAVFGLGSSMYPRFCAFAHDIDQKLSHLGASQLTPMGEGDELSGQEDAFRSWAVQTFKAACETFDVRGKQHIQIPKLYTSNVTWDPHHYRLVQDSQPLDLSKALSSMHAKNVFTMRLKSRQNLQSPTSSRATILVELSCEDGQGLNYLPGEHLGVCPGNQPALVQGILERVVDGPTPHQTVRLEALDESGSYWVSDKRLPPCSLSQALTYFLDITTPPTQLLLQKLAQVATEEPERQRLEALCQPSEYSKWKFTNSPTFLEVLEEFPSLRVSAGFLLSQLPILKPRFYSISSSRDHTPTEIHLTVAVVTYHTRDGQGPLHHGVCSTWLNSLKPQDPVPCFVRNASGFHLPEDPSHPCILIGPGTGIAPFRSFWQQRLHDSQHKGVRGGRMTLVFGCRRPDEDHIYQEEMLEMAQKGVLHAVHTAYSRLPGKPKVYVQDILRQQLASEVLRVLHKEPGHLYVCGDVRMARDVAHTLKQLVAAKLKLNEEQVEDYFFQLKSQKRYHEDIFGAVFPYEAKKDRVAVQPSSLEMSAL

SEQ ID NO: 12 - protein, 826 aa, Homo sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 13 - protein, 1016 aa, Homo sapiens
MKMASTRCKLARYLEDLEDVDLKKFKMHLEDYPPQKGCIPLPRGQTEKADHVDLATLMIDFNGEEKAWAMAVWIFAAINRRDLYEKAKRDEPKWGSDNARVSNPTVICQEDSIEEEWMGLLEYLSRISICKMKKDYRKKYRKYVRSRFQCIEDRNARLGESVSLNKRYTRLRLIKEHRSQQEREQELLAIGKTKTCESPVSPIKMELLFDPDDEHSEPVHTVVFQGAAGIGKTILARKMMLDWASGTLYQDRFDYLFYIHCREVSLVTQRSLGDLIMSCCPDPNPPIHKIVRKPSRILFLMDGFDELQGAFDEHIGPLCTDWQKAERGDILLSSLIRKKLLPEASLLITTRPVALEKLQHLLDHPRHVEILGFSEAKRKEYFFKYFSDEAQARAAFSLIQENEVLFTMCFIPLVCWIVCTGLKQQMESGKSLAQTSKTTTAVYVFFLSSLLQPRGGSQEHGLCAHLWGLCSLAADGIWNQKILFEESDLRNHGLQKADVSAFLRMNLFQKEVDCEKFYSFIHMTFQEFFAAMYYLLEEEKEGRTNVPGSRLKLPSRDVTVLLENYGKFEKGYLIFVVRFLFGLVNQERTSYLEKKLSCKISQQIRLELLKWIEVKAKAKKLQIQPSQLELFYCLYEMQEEDFVQRAMDYFPKIEINLSTRMDHMVSSFCIENCHRVESLSLGFLHNMPKEEEEEEKEGRHLDMVQCVLPSSSHAACSHGLVNSHLTSSFCRGLFSVLSTSQSLTELDLSDNSLGDPGMRVLCETLQHPGCNIRRLCNQKLVELDLSDNALGDFGIRLLCVGLKHLLCNLKKLWLVSCCLTSACCQDLASVLSTSHSLTRLYVGENALGDSGVAILCEKAKNPQCNLQKLGLVNSGLTSVCCSALSSVLSTNQNLTHLYLRGNTLGDKGIKLLCEGLLHPDCKLQVLELDNCNLTSHCCWDLSTLLTSSQSLRKLSLGNNDLGDLGVMMFCEVLKQQSCLLQNLGLSEMYFNYETKSALETLQEEKPELTVVFEPSW

SEQ ID NO: 14 - DNA, 270 bp, Artificial Sequence
  non-coding RNA gene for targeting antisense sequence of TXNIP promoter
gcgcacccgaacaacaaccattttccccgctaagagcacactgtgtccacgcgcctctgcggcctcgctgattggttagaggcctggtaaacaaggaccaagtagccaatgggagaactgtgcacgagggatgcacgagcctccgggccagcactcgcgtggagcgtcaagccaggcggctatataatgccgtttccggctcccgcttgacactctcctcttctggtctttggatatccagagttcctccagttgcgagaaaacagctgt

SEQ ID NO: 15 - DNA, 269 bp, Artificial sequence
  non-coding RNA gene for targeting sense sequence of TXNIP promoter
gcgggagccggaaacggcattatatagccgcctggcttgacgctccacgcgagtgctggcccggaggctcgtgcatccctcgtgcacagttctcccattggctacttggtccttgtttaccaggcctctaaccaatcagccaggccgcagaggcgcgtggacacagtgtgctcttagcggggaaaatggtagttgttcgggtgcgcagccttgccagggacgaggggcggggcctggggcagcgggtggctccctcgggagtctgggac

SEQ ID NO: 16 - DNA, 21 bp, Homo sapiens
aatggtcacgtcgaaatgaat

SEQ ID NO: 17 - DNA, 21 bp, Artificial sequence
  iRNA sense for TXNIP promoter target 1
uggucacgucgaaaugaautt

SEQ ID NO: 18 - DNA, 21 bp, Artificial sequence
  iRNA antisense for TXNIP promoter target 1
auucauuucgacgugaccatt

SEQ ID NO: 19 - DNA, 21 bp, Homo sapiens
aactgtgcacgagggatgcac

SEQ ID NO: 20 - DNA, 21 bp, Artificial sequence
  iRNA sense for TXNIP promoter target 2
cugugcacgagggaugcactt

SEQ ID NO: 21 - protein, 27 aa, Artificial sequence
  TP
GWTLNSAGYLLGKINLKALAALAKKIL

SEQ ID NO: 22 - protein, 21 aa, Artificial sequence
  TP10
AGYLLGKINLKALAALAKKIL

SEQ ID NO: 23 - protein, 18 aa, Artificial sequence
  pVEC
LLIILRRRIRKQAHAHSK

SEQ ID NO: 24 - protein, 16 aa, Artificial sequence
  Penetratin
RQIKIWFQNRRMKWKK

SEQ ID NO: 25 - protein, 14 aa, Artificial sequence
  Tat fragment
GRKKRRQRRRPPQC

SEQ ID NO: 26 - protein, 18 aa, Artificial sequence
  signal sequence based peptide
GALFLGWLGAAGSTMGAW

SEQ ID NO: 27 - protein, 18 aa, Artificial sequence
  amphiphilic model peptide
KLALKLALKALKAALKLA

SEQ ID NO: 28 - DNA, 4196 bp, Homo sapiens
gtggctcttctggcccgggctactatatagagacgtttccgcctcctgcttgaaactaacccctctttttctccaaaggagtgcttgtggagatcggatcttttctccagcaattgggggaaagaaggctttttctctgaattagcttagtgtaaccagcggcgtatattttttaggcgccttttcgaaaacctagtagttaatattcatttgtttaaatcttattttatttttaagctcaaactgcttaagaataccttaattccttaaagtgaaataattttttgcaaaggggtttcctcgatttggagctttttttttcttccaccgtcatttctaactcttaaaaccaactcagttccatcatggtgatgttcaagaagatcaagtcttttgaggtggtctttaacgaccctgaaaaggtgtacggcagtggcgagaaggtggctggccgggtgatagtggaggtgtgtgaagttactcgtgtcaaagccgttaggatcctggcttgcggagtggctaaagtgctttggatgcagggatcccagcagtgcaaacagacttcggagtacctgcgctatgaagacacgcttcttctggaagaccagccaacaggtaagcggcccaattcattgttggagggtgaaagctgattagagaagagaattgaatacacaaaacctgtacgaaatgttttaagttgctcagtttgagtggtttgaattacgtgttgttgcttccttttttctgttttaatttgcagacattctcctccccccccaaaaaaaagggtgatttgtacaattttttatggtgctgtgtcctaaaggggatcctgaggggcgttgcctcgggtagttaaagtcttatgtgtgcataagttgcttattctttgtctacttcctatttgagatgttagtagagaactgtcctgggtgaatctttcagtattgcagggcttggcaacttgctgcccgacaaaatacatcagaatttctctttaagaacaatatgggatggattaaaaaatatatatatgggatgaaattgggggtacttcaataccttgcatgccacccaagcattccttatcacacagatgcattttaagtgtaacagcaagcctaatggctactcgattttctttcccttcaggtgagaatgagatggtgatcatgagacctggaaacaaatatgagtacaagttcggctttgagcttcctcaggggtaaatatcagctaaatgcatctttgaacttttctgtctaaaatatcttgccctcctttgatcacttactgttcttggagagcgttttaaaattttcattttcttgacacaggcctctgggaacatccttcaaaggaaaatatgggtgtgtagactactgggtgaaggcttttcttgaccgcccgagccagccaactcaagagacaaagaaaaactttgaagtagtggatctggtggatgtcaatacccctgatttaatggtgagatccattttaaagattctattctcctgggtcatgaggatgataaatcagacgtcttgggcattagattgaagcatctcaaaacacctttttatccccttctatataggcacctgtgtctgctaaaaaagaaaagaaagtttcctgcatgttcattcctgatgggcgggtgtctgtctctgctcgaattgacagaaaaggattctgtgaaggtaaaatcctagtgcttatggcagttaaaacaggaactgggtttaggggcaagggcatgggactggggtagaggaagtgtcattaactatactgagtatcagtaagttttcatctttctcattgctaggtgatgagatttccatccatgctgactttgagaatacatgttcccgaattgtggtccccaaagctgccattgtggcccgccacacttaccttgccaatggccagaccaaggtgctgactcagaagttgtcatcagtcagaggcaatcatattatctcagggacatgcgcatcatggcgtggcaagagccttcgggttcagaagatcaggccttctatcctgggctgcaacatccttcgagttgaatattccttactggtgggtagatgcagggtggcttcagcagagaaattgttcatctgttaccacagctgtcttgtttctccagacctctggtgtaataaactgccatcttcctttctagatctatgttagcgttcctggatccaagaaggtcatccttgacctgcccctggtaattggcagcagatcaggtctaagcagcagaacatccagcatggccagccgaaccagctctgagatgagttgggtagatctgaacatccctgataccccagaaggtgagccagacctaatgtcttttctttgtttctggtctacctgggtttgtaaaattgtgatggtccagcatttcttgggcaggattcttatgtggccatattttcttttctagctcctccctgctatatggatgtcattcctgaagatcaccgattggagagcccaacactcctctgctagatgacatggatggctctcaagacagccctatctttatgtatgcccctgagttcaagttcatgccaccaccgacttatactgaggtgaggattgtcatctttactgttaaatttgtcctaagctttctataagaagttgacttagacggattgctaaactggtttgttctttttgttcttacctgaactgaaatagtctgtttctttctttaggtggatccctgcatcctcaacaacaatgtgcagtgagcatgtggaagaaaagaagcagctttacctacttgtttctttttgtctctcttcctggacactcactttttcagagactcaacagtctctgcaatggagtgtgggtccaccttagcctctgacttcctaatgtaggaggtggtcagcaggcaatctcctgggccttaaaggatgcggactcatcctcagccagcgcccatgttgtgatacaggggtgtttgttggatgggtttaaaaataactagaaaaactcaggcccatccattttctcagatctccttgaaaattgaggccttttcgatagtttcgggtcaggtaaaaatggcctcctggcgtaagcttttcaaggttttttggaggctttttgtaaattgtgataggaactttggaccttgaacttacgtatcatgtggagaagagccaatttaacaaactaggaagatgaaaagggaaattgtggccaaaactttgggaaaaggaggttcttaaaatcagtgtttcccctttgtgcacttgtagaaaaaaaagaaaaaccttctagagctgatttgatggacaatggagagagctttccctgtgattataaaaaaggaagctagctgctctacggtcatctttgcttagagtatactttaacctggcttttaaagcagtagtaactgccccaccaaaggtcttaaaagccatttttggagcctattgcactgtgttctcctactgcaaatattttcatatgggaggatggttttctcttcatgtaagtccttggaattgattctaaggtgatgttcttagcactttaattcctgtcaaattttttgttctccccttctgccatcttaaatgtaagctgaaactggtctactgtgtctctagggttaagccaaaagacaaaaaaaattttactacttttgagattgccccaatgtacagaattatataattctaacgcttaaatcatgtgaaagggttgctgctgtcagccttgcccactgtgacttcaaacccaaggaggaactcttgatcaagatgcccaaccctgtgatcagaacctccaaatactgccatgagaaactagagggcaggtcttcataaaagccctttgaacccccttcctgccctgtgttaggagatagggatattggcccctcactgcagctgccagcacttggtcagtcactctcagccatagcactttgttcactgtcctgtgtcagagcactgagctccacccttttctgagagttattacagccagaaagtgtgggctgaagatggttggtttcatgtttttgtattatgtatctttttgtatggtaaagactatattttgtacttaaccagatatatttttaccccagatggggatattctttgtaaaaaatgaaaataaagtttttttaatggaaaaaaaaa

SEQ ID NO: 29 - DNA, 3901 bp, Mus musculus
gacactctcctcctctggtctcggggtttccagagtttctccagttgcggaagacagctgttatttttctcctgaaagcttttggcacagccggcaggctgaaacttccaggcaccttttggaaaagttgttagggtttgtttgaagctttctttacattttcgtttgggttttcaagccctgactttacggaggcgagctcttcgtttgctttgaagggttcttaaagatttttttcctctccggctttcgtttttcttgaacccactcggctcaatcatggtgatgttcaagaagatcaagtcttttgaggtggtcttcaacgaccccgagaaggtgtacggcagcggggagaaggtggccggacgggtaatagtggaagtgtgtgaagttacccgagtcaaagccgtcaggatcctggcttgcggcgtggccaaggtcctgtggatgcaagggtctcagcagtgcaaacagactttggactacttgcgctatgaagacacacttctcctagaagagcagcctacaggtactgctcccagcaggactgatggtgacttgggaggtctgtgggtcggggagggcaccactaaatgtttcgagttgttcgtttgaatggtttgaactgttggtccctatatttttttactttgtaattagcaagtttttcactacccttcacccccctagagtgatttgaacactttctgaggtactgtttcctgaaagtgttgtcttagctactacttaaagattaatgtatttgtggatttcgcaactttctgtccaagaaagtgctctgggatcttttcttccatagtgtaagagatgaaagtggaagtgaagtaaggtagtctactgcccaggcactcctcattgacgctttcaaaatgtaacaagaagcctaatggccccttgtctttgtttcccagcaggtgagaacgagatggtgatcatgaggcctggaaacaaatatgagtacaagttcggcttcgagcttcctcaagggtaggcatccaccgtgtgcaccttgcactcttatttctaagtcttccccctccattgatctcttacagttcttagccttaattttggttcattgttttgacacaggcccctgggaacatcctttaaaggaaaatatggttgcgtagactactgggtgaaggcttttctcgatcgccccagccagccaactcaagaggcaaagaaaaacttcgaagtgatggatctagtggatgtcaatacccctgacctaatggtgaggattttttgtttttgtttttaaaaaggttttaaaattcttcttggtcagggataataaattagatgcatgggggttgaaatatctcaaaacattatttccttttacacaggcaccagtgtctgccaaaaaggagaagaaagtttcctgcatgttcattcctgatggacgtgtgtcagtctctgctcgaattgacagaaaaggattctgtgaaggtaaaaacatactgcttcaaatgctagacaggatagccagaactgggggtgggggggttgggggtggtacggagagggtcgtagggtagaggcagaggaagtgctgttaacttgcatggctattcatacttcctcattttattttaactctaggtgatgacatctccatccatgctgactttgagaacacgtgttcccgaatcgtggtccccaaagcggctattgtggcccgacacacttaccttgccaatggccagaccaaagtgttcactcagaagctgtcctcagtcagaggcaatcacattatctcagggacttgcgcatcgtggcgtggcaagagcctcagagtgcagaagatcagaccatccatcctgggctgcaacatcctcaaagtcgaatactccttgctggtgagtgggtgagaagagagacaattacctggttacaaattcagtgctttctgtactcaacccatctaacaaactgccatcctcctctctagatctacgtcagtgtccctggctccaagaaagtcatccttgatctgcccctagtgattggcagcaggtctggtctgagcagccggacatccagcatggccagccggacgagctctgagatgagctggatagacctaaacatcccagataccccagaaggtaagctgcagccggataggttcgagttattttgatctgcttgggcttgtggagttggggtgacctggcatttatttcttagtcggacttctgacaccgttttctctcttcagctcctccttgctatatggacatcattcctgaagatcacagactagagagccccaccacccctctgctggacgatgtggacgactctcaagacagccctatctttatgtacgcccctgagttccagttcatgcccccacccacttacactgaggtgagaactgctattctcacagggtcaacattttgtcctaggccttttgaaggaagggttaatgtgggttttctacttaactaaaaaacctgaaaatttcctctctattccccttccaggtggatccgtgcgtccttaacaacaacaacaacaacaacaacgtgcagtgagcctgcaggaaatgaagcatctgtattagcgcatttctttctgcctctctgcttgaactccagtgtttcagagactcagtctctacagcggggaacgggtacaccccagccgctgactcctcaagatgggtggcaatcagtaggcgggtctccggcttcaagtggtgcagaccagtgcccgcactgtggcataggagtgtttgctgggtggatgtcagaacactcttagaaaaattgagacctgaccactttctcggatgttggaaatgaagaacttgtttgtgttgactgagtcagggcactgctgaccttctggcgttgtctttccaaggtttttgttttaaagggacttttaaattgtctaaaatatcagtagaccatcatctgtgccatgggggacagagccaatttcaagtcatggccaaaattttgtaagaggagtgtttttgtgtgttttttaaagtcagtgttccttttttatatctttacaaagaaaagaccttccacggctggtgagcacgcagcctgtgaaattcggggcagctgctccaagttgacttcaccctgggagcagtagtagctgtgcccactgacggccataaaagccattttacagccagttgcactgtgttctcttgtaagcataatcagatgggagaatctgttatttccctgtaaccccttggaattgattctaaggtgatgttcttagcactttagcttgtcaattttgttttagtctccgttatagatgtaagctccaccagtctcttaaggattaagcccagtgacttggagggtgggggttagggtctctatccctgaacattgtagacccaggctggcctgagagatccacctgcctctgcctcctgagtgctgcgatcaaaggcccagcttggttattgcttttgaggctttctcccaacgcacagacttgtgtaattctaacactaatcctgtgaagggttgtggttgacagctggagcctgggtgacattctacattgagatgccccagcactgatcggggcacagaagcccccagaccccatttcctgtccagtgttgggagaaagtgctgctttcactgtggcctcagccctggctcggaagctcactaagccttagcactttgtcctgtgtcagctccacctgagaactgtgcagccagaatgtctgcgagctgatggaggtttcggttttgttgtttttgtattttgtgtatctttttgtatgattaaaaactatattttctacttatccaaatatattttcaccccaaagtggggttatcctttgtaaaaaaaaataaagttttttaatgac

SEQ ID NO: 30 - DNA, 4170 bp, Macaca mulatta
ctttctctgtcaagaagtgcttgcggagatcggatctcttctccagcaatcggggaaaagaagactttttctctgaaatcgcttagtgtaaccagcggcgtaaattttttggacgccttttcgaaaacctagttaaaattcatttgtttaaatctcattttatttttaagctcaaactgcttaagaatatcttcattccttaggaagagtgaaataatttactgcaaaggaatttcctcgatttggtgcatttttttttttattcttccacggtcttttctaactctcaaaccaactccgtcccatcatggtgatgttcaagaagatcaagtcttttgaggtggtctttaacgaccctgaaaaggtgtacggcagtggtgagaaggtggctggccgggtgatagtggaggtgtgtgaagttactcgtgtcaaagccgttaggatccttgcttgcggagtggctaaagtgctctggatgcagggatcccagcagtgcaaacagacttcggagtacctgcgctatgaagacacgcttcttccggaagaccagccaacaggtaagcggcccaattcattgttggagggtgaaagctgattagagaagagaattgaaaggaatacacaaaactatacgaaatgttttaagttgctcagtttgagtggtgtgaattgcacgttgttacttccttttttctaatttgcagacattctccgccccacaccccccaaataaaagggtggtttgtacaattttttatggtgctgtgtcctaaaggtggttctgaggggcattgcctcggatagttaaaaatcttatgtgtgcattagttgcttattctttgcctacttcctgtttgagatgttagtgaagaactgtcctgggtgaatctttaaaagcattgcagggcaacctgggaacttgctgcccaacaaaatacatcagaatttctctttaagaacaacatgggatgattaaaatacctctctgtgggacgaaattaggggtacttcaataccttgcatgccacccaaacattccttatcacacagatgcattttaagtgtaacaggaagcctaatggctactcgattttctttcccttcaggtgagaatgagatggtgatcatgagacctggaaacaaatatgagtacaagttcggctttgagcttccccaggggtaaatatcagctaaatgcatctttgaacttttctgtctaaaatatcttgccctcctttgatcacttactgttccttggagagcatttaaaaattttcattttcttgacacaggcctctgggaacatccttcaaaggaaaatatgggtgtgtagactactgggtgaaggcttttcttgaccgccccagccagccaactcaggagacaaagaaaaactttgaagtagtggatctggtggatgtcaatacccctgatttaatggtgagattcattttaaagattatattctcctgggtcacagggatgataaatcagatgtcttgggcattagattgaagcatctcaaaacacctttttgtccccttctacataggcacctgtgtctgctaaaaaggaaaagaaagtttcctgcatgttcattcctgatgggcgggtgtctgtctctgctcgaattgacagaaaaggattctgtgaaggtaaaatcctagtgcttatggcagacagatataacaggaactaggtttaggggcaagggcatgggactggggtagaggaaatgtcattaactatactgagtatcagtaagttttcatttttctctttgctaggtgatgagatttccatccatgctgactttgagaatacgtgttcccgaattgtggtccccaaagctgccattgtggcccgccacacttaccttgccaatggccagaccaaggtgctgactcagaagttgtcatcagtcagaggcaatcatattatctcagggacatgtgcatcatggcgtggaaagagccttcgggttcagaagatcaggccttctatcctgggctgcaacattcttcgagttgaatattctttactggtgagtagatgcagggtgggttcagcagagaaattgttcatctgttaccataactgtcttgtttcaccagagcgctctggtctaacaaactaccatcttccttttctagatctatgttagcgttcctgggtccaagaaggtcatccttgacctgcccctggtaattggcagcagatcaggtctaagcagcagaacatccagcatggccagccgaaccagctctgagatgagttgggtagatctgaatatccctgataccccagaaggtgagccagacctaatgtcttttcttcatttctggtctacttgggtttgtaaaattgtgatggtccagcatttcttgggcaggcttcttatgtggccatattttcttttctagctcctccctgctatatggatatcattcccgaagatcaccgattggagagccccaccactcctctgctagatgacatggatggcgctcaagacagccctatctttatgtatgcccctgagttcaagttcatgccaccacctacttatactgaggtgagaattgtcatatttactgttaaatttgtcctaagctttctataagaagttgacttagagggattgctaaactggtttgttcttttagttcttacctgaactgaaatagtctgtttcttcctttaggtggatccgtgcatcctcaacaacaacgtgcagtgaacatgtggaagaaaagaagcagctttacctacttctttctttttgtctctcttcctggactctcactttttcagagactcagtagtctctgcaatggagtgtggatccaccttagcctctgacttcccaatgcaggaggtggtcagcaggcaatctcctgggccttaaaggatgcggactcatcctcagccagcgccgatgttgtgatacaggggtgtttgttggatgggtttaaaaacagctagaaaaactcaggcccatccattttctcagatctccttgaaaattgaggccttttcggtagtttcgggtcaggtaaaaatggcctcctggcatgagcttttcaaggtttcttggaggctttgtgcaaattgtgataaccactttggaccttcaacttatgtatctatcatgtggaaaagagccaatttagcaaactaggaacatgaaaagggaaattatggccaaaactttgggaaaaggaggttcttaaaatcagtattcccctttgtgcacttgcagaaaaaaaagaaaaaccttctagggctgatttgatggacatttggagagagctttctctgtgattataaaaaaagaagctagctgctctatgggtcgtctttgcttagagtgtactttaacctggcttttaaagcggtagtaactgtgtcccaccaaaggtcttaaaagccatttttggagcgtattgcactgtgttctcctattgcaaatattttcatatgggaggatatttttctcttcatgcaagtccttggaattgattctaaggtgatgttcttagcactttagttcctgtcaaattttttgtcctcccccctccaccatcttaaatgtaagctgaaactcgtctactatgtctctaggggtaagcccaaagacaaaaagaaaaaaaaactgttaactacttttgagattgccccaatgtacagaattacataattctaagactcaaatcatgtgaaagggttgctgctgtcagccttgcccactgtgacttctccaaacccaaggaggacctcttgatcaaaatgcccaacactgatcagaacctccaaatactgccatgagaaactagagggcaggtcttcacaaaagcccttcgaacccccttcctgccctgtgttaggagatagggatattggcccctctctcactgcagctgccagcacttggtcagtcactctcagccatagcactttgttcactgtcctgtgtcagagcactgagctccacccttttctgagcgttactgcagccagaaagtgtggactgaaggtggtttcatgtttttgtattatgtatctttttgtatggtaaagactatattttgtacttcaccagatatatttttaccccagatggggatattcttgtaaaaaataaaaataaagtttttttaacgggaaaaa

SEQ ID NO: 31 - DNA, 1939 bp, Artificial sequence
  construct with TXNIP promoter and Trx-1 cDNA
tcgcgaacgcctaataattctgcccaaatatggaaggaggctaggactcaatgacaaggctctggcggggtggaggtgggaggaggtgggtgaggaatagggtaggggccctggggtgtcagggagtggtgggtagtccttcaacatacaagagtttctttgcactcaagttatttctctagtcagctcctgaagcatttctcagcaaggtttgccaaatagccaagtgaaaccaatacagcttcagccctggggagactgaaacaggctgaggggtatgcatttcatttagtgattttgatgagaggacaaatggggaaaaaaaaaaagagtgacaggaactcgggaacaaagtaaggagtgacagatcgttttcttcctttttcttcccccctgtttgtttgtttgtacccaccccttgtttcctggaggagcagggacgaagggagagatcagtgtaaaggtacacacctcactaaagctacagtgaggtaaataagggaacatatacaaaatgttccccaacctcacaggtacactgaagagatgaggggataagcaacaggatgtggacactcccttactgcttccgttccagagaacagaacagaatagaacgtaatgggcgaggaacaatagcagcacatagggcatggaacgaaggggtacacaccagaccatgtaccaataaggactttaagccagacttaaaatatctgacaagtcccccgcccccgggagatggaagcgttttattcaatagaagtgtaatgggagcacatcgggtaggctctttctcactaacacaactgcactctcgccctccgctccatcctgaagtatccttggggattgtttttcacagacttgcgaacttgtgagccaggaataaatggtcacgtcgaaatgaattgcgctggctaagacaggcatgaaatcctctcctaagcacatttttcttttacctaaaaaaagaaggggaaaaaaaccaacaaagcacacacccaaacaacccagctcccgagaggagtaccctggatgaggttcagggtctcggggtcccagactcccgagggagccacccgctgccccaggccccgcccctcctccctggcaaggctgcgcacccgaacaacaaccattttccccgctaagagcacactgtgtccacgcgcctctgcggcctcgctgattggttagaggcctggtaaacaaggaccaagtagccaatgggagaactgtgcacgagggatgcacgagcctccgggccagcactcgcgtggagcgtcaagccaggcggctatataatgccgtttccggctcccgcttgacactctcctcttctggtctttggatatccagagttcctccagttgcgagaaaacagctgttatttttctcctgaaagcttttggcacaaccagctggttgaaactttcaggcaccttttagagaagttgttaaggttttgtttgaggctttctttgggtttttaagccctctctgcctcacggagagacttaagttcttagtttgctgagaaggattctgaagagtttttcctctccggcttccgtttttcttgaacccactcggctcaatcatggtgaagctgatcgagagcaaggaagcttttcaggaggccctggccgctgcgggagacaagcttgtggtagtggacttctctgccacgtggtgtggaccttgcaaaatgatcaagcccttctttcattccctctgtgacaagtattccaatgtggtgttccttgaagtagacgtggatgactgccaggatgttgctgcagactgtgaagtcaaatgcatgccgaccttccagttctataaaaagggtcaaaaggttggggagttctctggtgctaacaaggaaaagctcgaagccactattacggagtttgcctaatcatgctctgaagggccc

SEQ ID NO: 32 - DNA, 468 bp, Homo sapiens
agccctccaggacaggctgcatcagaagaggccatcaagcagatcactgtccttctgccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgagcccttggccctggaggggtccctgcagaagcgtggcattgtggaacaatgctgtaccagcatctgctccctctaccagctggagaactactgcaactagacgcagcccgcaggcagccccacacccgccgcctcctgcaccgagagagatggaataaagcccttgaaccagcaaaa

SEQ ID NO: 33 - DNA, 725 bp, Homo sapiens
cttcagaagcaatgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggtatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatgtaggaagaccctcctgaggagtgaagagtgacatgccaccgcaggatcctttgctctgcacgagttacctgttaaactttggaacacctaccaaaaaataagtttgataacatttaaaagatgggcgtttcccccaatgaaatacacaagtaaacattccaacattgtctttaggagtgatttgcaccttgcaaaaatggtcctggagttggtagattgctgttgatcttttatcaataatgttctatagaaaag

SEQ ID NO: 34 - DNA, 911 bp, Homo sapiens
gtacaaaaaagcagaagggccgtcaaggcccaccatgaacggcgaggagcagtactacgcggccacgcagctttacaaggacccatgcgcgttccagcgaggcccggcgccggagttcagcgccagcccccctgcgtgcctgtacatgggccgccagcccccgccgccgccgccgcacccgttccctggcgccctgggcgcgctggagcagggcagccccccggacatctccccgtacgaggtgccccccctcgccgacgaccccgcggtggcgcaccttcaccaccacctcccggctcagctcgcgctcccccacccgcccgccgggcccttcccggagggagccgagccgggcgtcctggaggagcccaaccgcgtccagctgcctttcccatggatgaagtctaccaaagctcacgcgtggaaaggccagtgggcaggcggcgcctacgctgcggagccggaggagaacaagcggaccgcacggcctacacgcgcgcacagctgctagagctggagaaggagttcctattcaacaagtacatctcacggccgcgccgggtggagctggctgtcatgttgaacttgaccgagagacacatcaagatctggttccaaaaccgccgcatgaagtggaaaaaggaggaggacaagaagcgcggcggcgggacagctgtcggggtggcggggtcgcggagcctgagcaggactgcgccgtgacctccggcgaggagcttctggcgctgccgccgccgccgccccccggaggtgctgtgccgcccgctgcccccgttgccgcccgagagggccgcctgccgcctggccttagcgcgtcgccacagccctccagcgtcgcgcctcggcggccgcaggaaccacgatgaggcctcatgggcccagctttcttgtac

SEQ ID NO: 35 - DNA, 421 bp, Homo sapiens
atggtgaagcagatcgagagcaagactgcttttcaggaagccttggacgctgcaggtgataaacttgtagtagttgacttctcagccacgtggtgtgggccttgcaaaatgatcaaccctttctttcattccctctctgaaaagtattccaacgtgatattccttgaagtagatgtggatgactgtcaggatgttgcttcagagtgtgaagtcaaatgcacgccaacattccagttttttaagaagggacaaaaggtgggtgaattttctggagccaataaggaaaagcttgaagccaccattaatgaattagtctaatcatgttttctgaaaacataaccagccattggctatttaaacttgtatttttttatttacaaaatataaatatgaagacataaccagttgccatctgcgtgac

SEQ ID NO: 36 - DNA, 590 bp, Homo sapiens
atggctcagcgacttcttctgaggaggttcctggcctctgtcatctccaggaagccctctcagggtcagtggccacccctcacttccaaagccctgcagaccccacaatgcagtcctggtggcctgactgtaacacccaacccagcccggacaatatacaccacgaggatctccttgacaacctttaatatccaggatggacctgactttcaagaccgagtggtcaacagtgagacaccagtggttgtggatttccacgcacagtggtgtggaccctgcaagatcctggggccgaggttagagaagatggtggccaagcagcacgggaaggtggtgatggccaaggtggatattgatgaccacacagacctcgccattgagtatgaggtgtcagcggtgcccactgtgctggccatgaagaatggggacgtggtggacaagtttgtgggcatcaaggatgaggatcagttggaggccttcctgaagaagctgattggctgacaagcagggatgagtcctggttcccttgcccgcgtgggaccccaatagaactcagcccttccatgccagcccttcctgctgcctccctc

SEQ ID NO: 37 - DNA, 1271 bp, Homo sapiens
ggggtttcctcgatttggagctttttttttcttccaccgtcatttctaactcttaaaaccaactcagttccatcatggtgatgttcaagaagatcaagtcttttgaggtggtctttaacgaccctgaaaaggtgtacggcagtggcgagaaggtggctggccgggtgatagtggaggtgtgtgaagttactcgtgtcaaagccgttaggatcctggcttgcggagtggctaaagtgctttggatgcagggatcccagcagtgcaaacagacttcggagtacctgcgctatgaagacacgcttcttctggaagaccagccaacaggtgagaatgagatggtgatcatgagacctggaaacaaatatgagtacaagttcggctttgagcttcctcaggggcctctgggaacatccttcaaaggaaaatatgggtgtgtagactactgggtgaaggcttttcttgaccgcccgagccagccaactcaagagacaaagaaaaactttgaagtagtggatctggtggatgtcaatacccctgatttaatggcacctgtgtctgctaaaaaagaaaagaaagtttcctgcatgttcattcctgatgggcgggtgtctgtctctgctcgaattgacagaaaaggattctgtgaaggtgatgagatttccatccatgctgactttgagaatacatgttcccgaattgtggtccccaaagctgccattgtggcccgccacacttaccttgccaatggccagaccaaggtgctgactcagaagttgtcatcagtcagaggcaatcatattatctcagggacatgcgcatcatggcgtggcaagagccttcgggttcagaagatcaggccttctatcctgggctgcaacatccttcgagttgaatattccttactgatctatgttagcgttcctggatccaagaaggtcatccttgacctgcccctggtaattggcagcagatcaggtctaagcagcagaacatccagcatggccagccgaaccagctctgagatgagttgggtagatctgaacatccctgataccccagaagctcctccctgctatatggatgtcattcctgaagatcaccgattggagagcccaaccactcctctgctagatgacatggatggctctcaagacagccctatctttatgtatgcccctgagttcaagttcatgccaccaccgacttatactgaggtggatccctgcatcctcaacaacaatgtgcagtgagcatgtggaagaaaagaagca

SEQ ID NO: 38 - DNA, 990 bp, Homo sapiens
cagtgtgctggcggcccggcgcgagccggcccggccccggtcgggcctccgaaaccatgaactttctgctgtcttgggtgcattggagcctcgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacaccgcggccgccagcacactg

SEQ ID NO: 39 - DNA, 4205 bp, Homo sapiens
ataactttgtagcgagtcgaaaactgaggctccggccgcagagaactcagcctcattcctgctttaaaatctctcggccacctttgatgaggggactgggcagttctagacagtcccgaagttctcaaggcacaggtctcttcctggtttgactgtccttaccccggggaggcagtgcagccagctgcaagccccacagtgaagaacatctgagctcaaatccagataagtgacataagtgacctgctttgtaaagccatagagatggcctgtccttggaaatttctgttcaagaccaaattccaccagtatgcaatgaatggggaaaaagacatcaacaacaatgtggagaaagccccctgtgccacctccagtccagtgacacaggatgaccttcagtatcacaacctcagcaagcagcagaatgagtccccgcagcccctcgtggagacgggaaagaagtctccagaatctctggtcaagctggatgcaaccccattgtcctccccacggcatgtgaggatcaaaaactggggcagcgggatgactttccaagacacacttcaccataaggccaaagggattttaacttgcaggtccaaatcttgcctggggtccattatgactcccaaaagtttgaccagaggacccagggacaagcctacccctccagatgagcttctacctcaagctatcgaatttgtcaaccaatattacggctccttcaaagaggcaaaaatagaggaacatctggccagggtggaagcggtaacaaaggagatagaaacaacaggaacctaccaactgacgggagatgagctcatcttcgccaccaagcaggcctggcgcaatgccccacgctgcattgggaggatccagtggtccaacctgcaggtcttcgatgcccgcagctgttccactgcccgggaaatgtttgaacacatctgcagacacgtgcgttactccaccaacaatggcaacatcaggtcggccatcaccgtgttcccccagcggagtgatggcaagcacgacttccgggtgtggaatgctcagctcatccgctatgctggctaccagatgccagatggcagcatcagaggggaccctgccaacgtggaattcactcagctgtgcatcgacctgggctggaagcccaagtacggccgcttcgatgtggtccccctggtcctgcaggccaatggccgtgaccctgagctcttcgaaatcccacctgaccttgtgcttgaggtggccatggaacatcccaaatacgagtggtttcgggaactggagctaaagtggtacgccctgcctgcagtggccaacatgctgcttgaggtgggcggcctggagttcccagggtgccccttcaatggctggtacatgggcacagagatcggagtccgggacttctgtgacgtccagcgctacaacatcctggaggaagtgggcaggagaatgggcctggaaacgcacaagctggcctcgctctggaaagaccaggctgtcgttgagatcaacattgctgtgctccatagtttccagaagcagaatgtgaccatcatggaccaccactcggctgcagaatccttcatgaagtacatgcagaatgaataccggtcccgtgggggctgcccggcagactggatttggctggtccctcccatgtctgggagcatcacccccgtgtttcaccaggagatgctgaactacgtcctgtcccctttctactactatcaggtagaggcctggaaaacccatgtctggcaggacgagaagcggagacccaagagaagagagattccattgaaagtcttggtcaaagctgtgctctttgcctgtatgctgatgcgcaagacaatggcgtcccgagtcagagtcaccatcctctttgcgacagagacaggaaaatcagaggcgctggcctgggacctgggggccttattcagctgtgccttcaaccccaaggttgtctgcatggataagtacaggctgagctgcctggaggaggaacggctgctgttggtggtgaccagtacgtttggcaatggagactgccctggcaatggagagaaactgaagaaatcgctcttcatgctgaaagagctcaacaacaaattcaggtacgctgtgtttggcctcggctccagcatgtaccctcggttctgcgcctttgctcatgacattgatcagaagctgtcccacctgggggcctctcagctcaccccgatgggagaaggggatgagctcagtgggcaggaggacgccttcgcagctgggccgtgcaaaccttcaaggcagcctgtgagacgtttgatgtccgaggcaaacagcacattcagatccccaagctctacacctccaatgtgacctgggacccgcaccactacaggctcgtgcaggactcacagcctttggacctcagcaaagccctcagcagcatgcatgccaagaacgtgttcaccatgaggctcaaatctcggcagaatctacaaagtccgacatccagccgtgccaccatcctggtggaactctcctgtgaggatggccaaggcctgaactacctgccgggggagcaccttggggtttgcccaggcaaccagccggccctggtccaaggtatcctggagcgagtggtggatggccccacaccccaccagacagtgcgcctggaggccctggatgagagtggcagctactgggtcagtgacaagaggctgcccccctgctcactcagccaggccctcacctacttcctggacatcaccacacccccaacccagctgctgctccaaaagctggcccaggtggccacagaagagcctgagagacagaggctggaggccctgtgccagccctcagagtacagcaagtggaagttcaccaacagccccacattcctggaggtgctagaggagttcccgtccctgcgggtgtctgctggcttcctgctttcccagctccccattctgaagcccaggttctactccatcagctcctcccgggatcacacgcccacagagatccacctgactgtggccgtggtcacctaccacacccgagatggccagggtcccctgcaccacggcgtctgcagcacatggctcaacagcctgaagccccaagacccagtgccctgctttgtgcggaatgccagcggcttccacctccccgaggatccctcccatccttgcatcctcatcgggcctggcacaggcatcgcgcccttccgcagtttctggcagcaacggctccatgactcccagcacaagggagtgcggggaggccgcatgaccttggtgtttgggtgccgccgcccagatgaggaccacatctaccaggaggagatgctggagatggcccagaagggggtgctgcatgcggtgcacacagcctattcccgcctgcctggcaagcccaaggtctatgttcaggacatcctgcggcagcagctggccagcgaggtgctccgtgtgctccacaaggagccaggccacctctatgtttgcggggatgtgcgcatggcccgggacgtggcccacaccctgaagcagctggtggctgccaagctgaaattgaatgaggagcaggtcgaggactatttctttcagctcaagagccagaagcgctatcacgaagatatctttggtgctgtatttccttacgaggcgaagaaggacagggtggcggtgcagcccagcagcctggagatgtcagcgctctgagggcctacaggaggggttaaagctgccggcacagaacttaaggatggagccagctctgcattatctgaggtcacagggcctggggagatggaggaaagtgatatcccccagcctcaagtcttatttcctcaacgttgctccccatcaagccctttacttgacctcctaacaagtagcaccctggattgatcggagcctcctctctcaaactggggcctccctggtcccttggagacaaaatcttaaatgccaggcctggcaagtgggtgaaagatggaacttgctgctgagtgcaccacttcaagtgaccaccaggaggtgctatcgcaccactgtgtatttaactgccttgtgtacagttatttatgcctctgtatttaaaaaactaacacccagtctgttccccatggccacttgggtcttccctgtatgattccttgatggagatatttacatgaattgcattttactttaatcaca

SEQ ID NO: 40 - DNA, 4081 bp, Homo sapiens
gcgcgcgccggcctgggcaggcgagcgggcgcgctcccgccccctctcccctccccgcgcgcccgagcgcgcctccgcccttgcccgccccctgacgctgcctcagctcctcagtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaatttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccaggcaaaaaaaaaaaaaaaaaaaaaaa

SEQ ID NO: 41 - DNA, 3207 bp, Homo sapiens
tgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacactgtggtgttccagggggcggcagggattgggaaaacaatcctggccaggaagatgatgttggactgggcgtcggggacactctaccaagacaggtttgactatctgttctatatccactgtcgggaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatggattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattaggagattgtgcaaccagaagctggtggagctggacctgagtgacaacgccctcggtgacttcggaatcagacttctgtgtgtgggactgaagcacctgttgtgcaatctgaagaagctctggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaa

SEQ ID NO: 42 - DNA, 21 bp, Artificial sequence
  iRNA antisense for TXNIP target 2
gugcaucccucgugcacagtt

SEQ ID NO: 43 - DNA, 336 bp, Homo sapiens
atggtgaagctgatcgagagcaaggaagcttttcaggaggccctggccgctgcgggagacaagcttgtggtagtggacttctctgccacgtggtgtggaccttgcaaaatgatcaagcccttctttcattccctctgtgacaagtattccaatgtggtgttccttgaagtagacgtggatgactgccaggatgttgctgcagactgtgaagtcaaatgcatgccgaccttccagttctataaaaagggtcaaaaggttggggagttctctggtgctaacaaggaaaagctcgaagccactattacggagtttgcctaatcatgctctgaagggccc

SEQ ID NO: 44 - protein, 247 aa, Homo sapiens
MTILFLTMVISYFGCMKAAPMKEANIRGQGGLAYPGVRTHGTLESVNGPKAGSRGLTSLADTFEHVIEELLDEDHKVRPNEENNKDADLYTSRVMLSSQVPLEPPLLFLLEEYKNYLDAANMSMMVLRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 45 - DNA, 1335 bp, Homo sapiens
ggggctgccgccgccgcgcccgggcgacccgcccgctcgctgtcccgcgcaccccgtagcgcctcgggctcccgggccggacagaggagcccggtgcgcccctccacctcctgctcggggggctttaatgagacacccaccgctgctgtggggccggcggggagcagcaccgcgacggggaccggggctgggcgctggagccagaatcggaaccacgatttgactccgccgccggggacccgtgagtttgtgtggaccccgagttccaccaggtgagaagagtgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccataaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgatggtcctgcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtggatttatgttgtatagattagattatattgagacaaaaattatctatttgtatatatacataacagggtaaattattcagttaagaaaaaaataattttattaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaatttaaaaaaaataatcccttgcatgctgccc

SEQ ID NO: 46 - protein, 211 aa, Homo sapiens
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 47 - DNA, 636 bp, Homo sapiens
atgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctga